(19) United States
(12) Patent Application Publication
(10) Pub. No.: US 2011/0135739 A1
(43) Pub. Date: Jun. 9, 2011

(54) Oral Formulations of a Hedgehog Pathway Inhibitor

(76) Inventors: Bennett Carter, Stoughton, MA (US); John J. Lee, Arlington, MA (US); Hana Sheikh, North Andover, MA (US)

(21) Appl. No.: 12/941,987

(22) Filed: Nov. 8, 2010

Related U.S. Application Data
(60) Provisional application No. 61/280,628, filed on Nov. 6, 2009.

(51) Int. Cl. A61K 9/14; C07D 221/20; A61K 31/438; A61P 35/00

(52) U.S. Cl. 424/489; 546/15; 514/278

(57) ABSTRACT

Oral formulations of the drug product IPI-926 are described. Pharmaceutical formulations (e.g., solid dosage forms) that are useful for the oral administration of a compound of formula (I), or a pharmaceutically acceptable salt thereof (e.g., IPI-926), to a human or animal subject are disclosed.

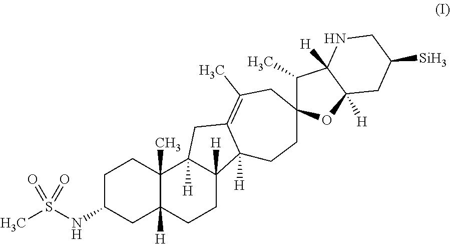

The formulations can further include, for example and without limitation, one or more other pharmaceutically-acceptable filler(s), binder(s), surfactant(s), and disintegrant(s); as well as one or more other therapeutic agent(s). Methods of preparing and using said formulations are also disclosed.

### CROSS REFERENCE TO RELATED APPLICATION

THIS APPLICATION CLAIMS THE BENEFIT OF U.S. PROVISIONAL APPLICATION No. 61/280,628, FILED ON Nov. 6, 2009, WHICH IS INCORPORATED HEREIN BY REFERENCE IN ITS ENTIRETY.

### BACKGROUND

Malignant activation of the hedgehog (Hh) pathway plays a pivotal role in cancer (Jiang and Hui, “Hedgehog Signaling in Development and Cancer” Developmental Cell Review (2008) 15:801-812). This pathway involves two cell-membrane proteins, Patched (PTC) and Smoothened (SMO), and is regulated by the absence or presence of Hh ligand. In most adult cells, the Hh pathway is not active. In the absence of ligand, Smo is held in an inactive state by Ptc, and Gli transcription factors do not enter the nucleus to promote transcription. When Hh ligand is present, it binds to Ptc, allowing Smo to initiate a signaling cascade that modifies Gli transcription factors and results in translocation of Gli into the nucleus. This leads to transcription of genes that promote tumor survival and growth. Because Smo plays a critical role in malignant activation of the Hh pathway, Smo is a target for the management of a broad range of cancers. IPI-926 is a novel, semisynthetic Hh pathway inhibitor that directly blocks the activity of Smo (Tremblay et al., “Discovery of a Potent and Orally Active Hedgehog Pathway Antagonist (IPI-926)” Journal of Medicinal Chemistry (2009) 52:14 4400-4418).

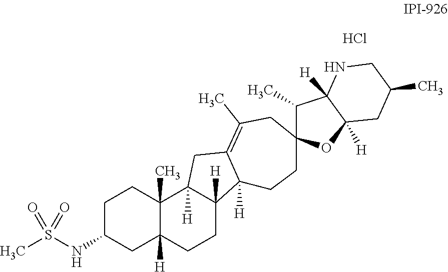

Oral administration is among the preferred routes for administration of pharmaceuticals since this route is generally convenient and acceptable to patients. In this type of administration, the drug substance typically needs to be absorbed through at least one membrane. In cases where the drug substance is part of a solid oral dosage form, absorption of the drug substance typically occurs once the solid oral dosage form is dissolved. The above can sometimes have considerable effects on drug pharmacokinetics and may cause a reduction in the actual amount of drug substance that is absorbed.

### SUMMARY

This application features pharmaceutical formulations (e.g., solid dosage forms) that are useful for the oral administration of a compound of formula (I) (shown below), or a pharmaceutically acceptable salt thereof (e.g., IPI-926), to a human or animal subject.

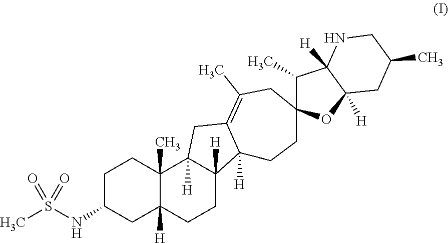

The formulations include the compound of formula (I), or a pharmaceutically acceptable salt thereof (e.g., IPI-926), as the active ingredient. The formulations can further include, for example and without limitation, one or more other pharmaceutically-acceptable filler(s), binder(s), surfactant(s), and disintegrant(s); as well as one or more other therapeutic agent(s). This application also features methods of preparing and using said formulations.

In general, for an orally administered drug to provide a therapeutic effect, the drug needs at least to be solubilized and then diffuse through the gut wall into the body. This process, however, can be impeded when a drug has, for example, low solubility and/or other features that confer poor dissolution properties. When the drug or formulation is a solid, this problem is typically addressed by reducing the particle size of the drug. Reducing the particle size of a solid increases the solid's surface area per gram. In other words, reducing the particle size of a solid increases the amount of surface area that is available for dissolution. Generally, when a solid's surface area per gram increases, its dissolution rate will also increase. As such, reducing the particle size of a solid is expected to increase the dissolution rate for that particular solid.

IPI-926 has been observed to gel, rather than dissolve, when dissolution is attempted in various aqueous media. Gel formation is problematic from a formulation standpoint because it can result, for example, in irregular release of IPI-926 in vivo. Low rates of dissolution, and subsequent gel formation, persist even when small particle sizes (e.g., less than 150 micrometers) of IPI-926 are employed.

However, it has been surprisingly found that both the extent and rate of dissolution of IPI-926 can be enhanced (e.g., 75% dissolution after 90 minutes at 37° C. in a dissolution media selected from 0.1 N aqueous HCl and 0.1 N aqueous HCl/0.5% Tween) by increasing the particle size of the formulated IPI-926. This result was surprising and unexpected because it was expected that dissolution rates for larger particles tend to be lower than those for smaller particles. Again, this is because the larger particles tend to have a lower surface area per gram and therefore less surface area available for dissolution. In one implementation of the subject matter described herein, formulated IPI-926 having a particle size of greater than or equal to 500 micrometers was found to undergo dissolution at a practical rate (e.g., 75% dissolution after 90 minutes at 37° C. in a dissolution media selected from 0.1 N aqueous HCl and 0.1 N aqueous HCl/0.5% Tween) and do so without any substantial gel formation.

Further, it has been found that by balancing the distribution of large and small particle sizes within the formulations, one can still employ non-trivial amounts (e.g., 20-50%) of smaller sized particles, e.g., particle sizes of formulated IPI-926 known to gel upon attempted dissolution (e.g., less than 250 micrometers, less than 150 micrometers, less than 125 micrometers). Thus, one need not use only larger dimensioned particles in order to achieve the desirable results discussed above, which can be advantageous for scale-up purposes. Accordingly, in some implementations of the subject matter described herein, the particle size can vary throughout the formulations and can include, e.g., both relatively large (e.g., greater than or equal to 500 micrometers) and relatively small (e.g., less than 250 micrometers, less than 150 micrometers, less than 125 micrometers) sized particles. It has been further found that one can also use compound of varying crystallinity (e.g., more than 80% crystalline or less than 80% crystalline) and achieve the dissolution properties described above. For example, it has been found that compound that is, e.g., more than 80% crystalline, can be used by increasing the particle size of the formulated IPI-926 (e.g., at least 50% of particles of the formulation having a particle size of greater than 500 micrometers).

As used herein, the term “particle” refers to a solid composite that includes the compound of formula (I), or a pharmaceutically acceptable salt thereof, and if present, one or more additional pharmaceutically acceptable solids (e.g., one or more pharmaceutically-acceptable filler(s), binder(s), surfactant(s). and disintegrant(s); as well as one or more other therapeutic agent(s)). Likewise, the term “particle size,” refers to the size of a solid composite that includes the compound of formula (I), or a pharmaceutically acceptable salt thereof, and if present, one or more additional pharmaceutically acceptable solids (e.g., one or more pharmaceutically-acceptable filler(s), binder(s), surfactant(s). and disintegrant(s); as well as one or more other therapeutic agent(s)).

For ease of exposition, any solid substance described herein having a particle size of less than 250 micrometers, including (but not limited to) particle sizes of less than 150 micrometers or less than 125 micrometers, will be sometimes referred to herein as “fines” or “in the form of fines.”

The formulations described herein are preferably used to achieve enhanced post-delivery solubilization of the orally administered compound of formula (I), e.g., IPI-926. In some embodiments, the formulations can be used to achieve an enhancement in the extent and/or rate of dissolution of the compound of formula (I), e.g., IPI-926, in the stomach and gastrointestinal tract, thereby increasing the likelihood that the compound will be absorbed by these tissues prior to excretion and/or degradation of the compounds. As such, the oral bioavailability of the compounds can be enhanced when formulated as described herein. Further enhancements in solubility and/or rate of dissolution can also be attained when the compound of formula (I), e.g., IPI-926, is formulated as a wet granulated formulation. It has been found that the majority of compound obtained via wet granulation tends to exhibit, e.g., larger particle sizes and/or lower percent crystallinity and is further observed to dissolve at practical rates and without substantial detectable gel formation.

In one aspect, pharmaceutical formulations are featured, which include a compound of formula (I):

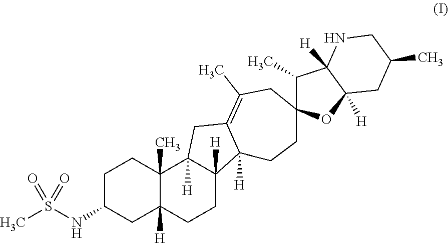

or a pharmaceutically acceptable salt thereof, in which the amount of fines that is present in the formulation does not cause gel formation; and in which the formulation is in a form that is suitable for oral administration. As used herein, the phrase “the amount of fines that is present in the formulation does not cause gel formation” is intended to include formulations that do not contain fines.

In another aspect, pharmaceutical formulations are featured, which include a compound of formula (I), or a pharmaceutically acceptable salt thereof, in which not more than 80% of the formulation have a particle size of less than 250 micrometers; and in which the formulation is in a form that is suitable for oral administration. As used herein, the phrase “not more than 80% of the formulation have a particle size of less than 250 micrometers” is intended to include formulations that do not contain particles that are less than 250 micrometers in size (i.e., 0% of the formulation having a particle size of less than 250 micrometers).

In a further aspect, pharmaceutical formulations are featured, which include a compound of formula (I), or a pharmaceutically acceptable salt thereof, in which not more than 80% of the formulation have a particle size of less than 150 micrometers; and in which the formulation is in a form that is suitable for oral administration. As used herein, the phrase “not more than 80% of the formulation have a particle size of less than 150 micrometers” is intended to include formulations that do not contain particles that are less than 150 micrometers in size (i.e., 0% of the formulation having a particle size of less than 150 micrometers).

In another aspect, pharmaceutical formulations are featured, which include a compound of formula (I), or a pharmaceutically acceptable salt thereof, in which not more than 60% of the formulation have a particle size of less than 125 micrometers; and in which the formulation is in a form that is suitable for oral administration. As used herein, the phrase “not more than 60% of the formulation have a particle size of less than 125 micrometers” is intended to include formulations that do not contain particles that are less than 125 micrometers in size (i.e., 0% of the formulation having a particle size of less than 125 micrometers).

Particle sizing and particle size determination were carried out as follows. Approximately 50-100 g of the dried granulation is removed after blending. The following United States standard sieves (described below in terms of screen mesh and pore size) were stacked from largest to smallest: 20 (850 μm), 40 (425 μm), 60 (250 μm), 80 (180 μm), 120 (125 μm), 200 (75 μm), and pan. The weighed granulation was then transferred to the #20 mesh sieves, which is located at the top of the stack. The sieves were then mechanically agitated in alternate fashion using vibrational and blunt force to agitate the sieves. The sieves were agitated until no more particles pass through any of the sieves. The weight of granulation retained on each screen is then determined. The % w/w retained on each screen is then determined using the following formulation:

(wt. of granulation on screen/total wt of granulation)*100.

Particle sizes can also be determined using other conventional methods known in the art, such as laser diffraction and light scattering can be used to determine particle size.

In one aspect, pharmaceutical formulations are featured, which include a compound of formula (I), or a pharmaceutically acceptable salt thereof, in which the compound of formula (I) or salt thereof is less than 20% crystalline (or less than 10% crystalline or less than 5% crystalline, e.g., 1, 2, 3, or 4% crystalline).

The % crystallinity was determined as follows. IPI-926 drug substance (“DS”) is both an isopropanol (“WA”) solvate and a crystalline solid. Since the IPA in the solvate is present in a one to one ratio with the active ingredient, the % crystallinity of the formulations described herein was determined based on the amount of residual IPA in the formulations as determined by gas chromatography (“GC”). While not wishing to be bound by theory, it is believed that IPA is (i) released when the crystal lattice of the pre-granulation IPI-926 drug substance is broken during granulation and (ii) subsequently removed upon drying of the formulation. As such, any IPA remaining in the formulation is therefore believed to be attributed to the presence of crystalline drug substance. A 50 milligram (“mg”) sample of pre-granulation, crystalline IPI-926 drug substance and a 50 mg sample of the formulation were each placed in separate vials, dissolved in 5.0 mL of DMSO, and assayed by GC. The residual IPA in the formulation was determined and expressed in parts per million (“ppm”). The amount of IPA in the pre-granulation drug substance was also determined and adjusted for the percentage of IPI-926 in the overall formulation. For example, if the amount of IPA in the drug substance was determined to be 100,000 ppm, and the formulation contained 10% of drug substance, then the theoretical IPA concentration of the formulation would be (100,000)(0.1)=10,000 ppm. The % crystallinity was determined using the following equation:

(ppm IPA in formulation/((% 926 in formulation/100)*ppm IPA in DS))*100=% crystallinity.

The degree of crystallinity can also be determined using conventional methods known in the art, e.g., differential scanning calorimetry, FTNIR, or microscopy.

In one aspect, pharmaceutical formulations are featured, which include a compound of formula (I), or a pharmaceutically acceptable salt thereof, in which when the formulation is stirred at 37° C. in a dissolution media selected from 0.1 N aqueous HCl and 0.1 N aqueous HCl/0.5% Tween and at an theoretical maximum concentration selected from 0.011 mg of the compound of formula (I)/mL of dissolution media, 0.033 mg of the compound of formula (I)/mL of dissolution media, and 0.133 mg of the compound of formula (I)/mL of dissolution media, dissolution of the compound of formula (I) is at least at least 60% (e.g., at least 65%, at least 70%, at least 75%, at least 80%, at least 85%, at least 90%, at least 95%, at least 98%; e.g., at least 75% complete) after 90 minutes as determined by HPLC.

The extent of dissolution was determined using the following dissolution/sampling conditions for formulations containing 10 mg or 30 mg of active ingredient:

| | |
|---|---|
| Dissolution medium | 0.1N HCl |
| Media volume | 900 milliliters (“mL”) |
| Temperature | 37 ± 0.5° C. |
| Apparatus | USP No. 2 (Paddles) |
| Speed | 75 rpm |
| Sampling Time | 10, 30, 60, 90 and 120 minutes |
| Infinity Point | 200 rpm for 30 minutes (150 minutes) |
| Sampling Volume | 5 mL (with 4 mL discard volume) |

The extent of dissolution was followed and determined by HPLC:

| | |
|---|---|
| Mobile Phase A: | 0.1% TFA in water |
| Mobile Phase B | 0.1% TFA in acetonitrile |
| Flow Rate | 1.5 mL/minute |
| Column | Waters Symmetry C18 |
| | 150 mm × 4.6 mm × 5 μm |
| Column Temperature | 40° C. |
| Autosampler Temperature | 5° C. |
| Injector volume | 75 μL |
| Detector Wavelength | 215 nm |
| Run time | 10.0 minutes |

The gradient program was as follows: 0 minutes (70% A/30% B); 6 minutes (35% A/65% B); 6.5 minutes (5% A/95% B); 7.0 minutes (70% A/30% B); 10.0 minutes (70% A/30% B).

The extent of dissolution for formulations containing 120 mg of active ingredient was determined using essentially the same conditions described above except that the dissolution medium contained 0.1 N aqueous HCl/0.5% Tween and the HPLC volume was 20 μL.

In one aspect, pharmaceutical formulations are featured, which include a compound of formula (I), or a pharmaceutically acceptable salt thereof, in which the formulations exhibit long term stability.

For example, the formulations are stable upon actual or simulated storage at 5° C. for at least 6 months (e.g., at least 9 months, at least 12 months, at least 18 months, at least 24 months).

As another example, the formulations are stable upon actual or simulated storage at 25° C./60% relative humidity for at least 3 months (e.g., at least 6 months, at least 9 months, at least 12 months, at least 18 months, at least 24 months).

As a further example, the formulations are stable upon actual or simulated storage at 40° C./75% relative humidity for at least 1 month (e.g., at least 2 months, at least 3 months, at least 6 months).

Stability studies were carried out as follows. Seven encapsulated formulations were placed in a 30 mL bottle, wide mouth, type III amber glass, cleaned (W015122) with rayon coil (28846) and fitted with a white polypropylene closure having a 0.040 thick F217 foamed polyethylene with 0.005 thick Teflon faced (W015122). The samples were stored in a controlled environment at the indicated temperature/relative humidity and tested for the following Appearance, HPLC Relative Retention Time, Purity, Impurities/Degradants, Assay, Moisture Content, Dissolution, and Microbial Limits Testing. The formulations were determined to be stable if Purity is ≧95%, Appearance fits the criteria: Intact size “x” capsule containing a white to off-white solid, HPLC Releative Retention time is 0.98-1.02, Assay is 90.0-110.0% of label claim, dissolution fits the USP <711> criteria Q ≧75% at 90 minutes, and Total yeast and mold ≦10² cfu/g and Total Aerobic Microbial Count ≦10³ cfu/g during microbial testing. All impurities ≧0.1% w/w are reported. Moisture content is for informational purposes.

In one aspect, pharmaceutical formulations are featured, which include a compound of formula (I), or a pharmaceutically acceptable salt thereof, in which administration of a single dose of the formulation to a beagle dog produces a mean peak plasma concentration (Cmax) of the compound of formula (I) of between 180 and 225 ng/mL for a formulation containing 30 mg of active ingredient (the compound of formula (I)); and /or 30 mg/day daily administration of the formulation to a beagle dog produces a mean steady state area under the concentration time curve (AUC_{(0-24 hsrs)}) of the compound of formula (I) of between 7000 and 10,000 nghr/mL, or between 8000 and 9500 nghr/mL. In another aspect, pharmaceutical formulations are featured, which include a compound of formula (I), or a pharmaceutically acceptable salt thereof, in which administration of a single dose of the formulation to a beagle dog produces a mean peak plasma concentration (Cmax) of the compound of formula (I) of between 60 and 80 ng/mL for a formulation containing 10 mg of active ingredient; and/or 10 mg/day daily administration of the formulation to a beagle dog produces a mean steady state area under the concentration time curve (AUC_{(0-24 hrs)}) of the compound of formula (I) of between 2000 and 3000 nghr/mL.

In some embodiments, a pharmaceutical formulation including a compound of formula (I), or a pharmaceutically acceptable salt thereof, when dosed at a dose of 1 mg/kg of active compound, are capable of delivering an amount of compound sufficient to achieve an AUC of at least 1000 ng·ml/hr, at least 5000 ng·ml/hr, or at least 10,000 ng·ml/hr of the active compound. In some embodiments, a pharmaceutical formulation including a compound of formula (I), or a pharmaceutically acceptable salt thereof, when dosed at a dose of 2 mg/kg of active compound, are capable of delivering an amount of compound sufficient to achieve an AUC of at least 5000 ng·ml/hr, at least 10,000 ng·ml/hr, or at least 15,000 ng·ml/hr, of the active compound. AUC values can be determined using conventional methods known in the art, see, e.g., Goodman and Gilman's The Pharmacological Basis of Therapeutics, 10th ed.; Hardman, J. G., Limbird, L. E., Eds.; McGraw-Hill: New York, 2001.

In one aspect, pharmaceutical formulations are featured, which include a compound of formula (I), or a pharmaceutically acceptable salt thereof, in which the formulation is in a form that is suitable for oral administration; and in which any two or more of the following features apply, or in which any three or more of the following features apply:
the amount of the compound of formula (I) or salt thereof that is present in the form of fines does not cause gel formation;
not more than 80% of the compound of formula (I) or salt thereof have a particle size of less than 250 micrometers;
not more than 80% of the compound of formula (I) or salt thereof have a particle size of less than 150 micrometers;
not more than 60% of the compound of formula (I) or salt thereof have a particle size of less than 125 micrometers;
the compound of formula (I) or salt thereof is less than 20% crystalline (or less than 10% crystalline or less than 5% crystalline);
when the formulation is stirred at 37° C. in a dissolution media selected from 0.1 N aqueous HCl and 0.1 N aqueous HCl/0.5% Tween and at an maximum concentration selected from 0.011 mg of the compound of formula (I)/mL of dissolution media, 0.033 mg of the compound of formula (I)/mL of dissolution media, and 0.133 mg of the compound of formula (I)/mL of dissolution media, dissolution of the compound of formula (I) is at least 75% complete after 90 minutes as determined by HPLC;
the formulations are stable upon actual or simulated storage at 5° C. for at least 6 months (e.g., at least 9 months, at least 12 months, at least 18 months, at least 24 months);
the formulations are stable upon actual or simulated storage at 25° C./60% relative humidity for at least 3 months (e.g., at least 6 months, at least 9 months, at least 12 months, at least 18 months, at least 24 months);
the formulations are stable upon actual or simulated storage at 40° C./75% relative humidity for at least 1 month (e.g., at least 2 months, at least 3 months, at least 6 months);
administration of a single dose of the formulation to a beagle dog produces a mean peak plasma concentration (Cmax) of the compound of formula (I) of between 180 and 225 ng/mL for a formulation containing 30 mg of active ingredient and/or between 60 and 80 ng/mL for a formulation containing 10 mg of active ingredient; and /or 30 mg daily administration of the formulation to a beagle dog produces a mean steady state area under the concentration time curve (AUC_{(0-24 hrs)}) of the compound of formula (I) of 7000 and 10,000 nghr/mL.
administration of the formulation containing 1 mg/kg of active compound of formula (I) to a human is capable of delivering an amount of compound sufficient to achieve an AUC of at least 1000 ng·ml/hr of the active compound.

In one aspect, oral pharmaceutical dosage formulations are featured, which include a compound of formula (I):

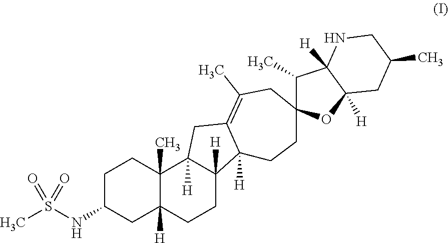

or a pharmaceutically acceptable salt thereof, wherein said compound is greater than 80% crystalline (e.g., greater than 85% crystalline, greater than 90% crystalline, greater than 95% crystalline, greater than 98% crystalline) and at least 50% (e.g., at least 60 percent, at least 70%, at least 80 percent, at least 90%)of particles of said formulation have a particle size of greater than 500 micrometers and wherein the formulation is in a form that is suitable for oral administration. In certain embodiments, at least 60% of particles of said formulation have a particle size of greater than 500 micrometers; at least 80% of particles of said formulation have a particle size of greater than 500 micrometers.

In one aspect, oral pharmaceutical dosage formulations are featured, which include a compound of formula (I):

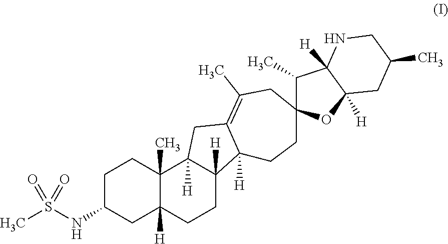

or a pharmaceutically acceptable salt thereof, wherein said compound is less than 80% crystalline (e.g., less than 70% crystalline, less than 60% crystalline, less than 50% crystalline, less than 40% crystalline, less than 30% crystalline, less than 20% crystalline) and at least 20% (e.g., at least 30 percent, at least 40 percent, at least 50 percent, at least 60 percent, at least 70%, at least 80 percent, at least 90%) of particles of said formulation have a particle size of greater than 250 micrometers and wherein the formulation is in a form that is suitable for oral administration. In certain embodiments, at least 40% of particles of said formulation have a particle size of greater than 250 micrometers; at least 50% of particles of said formulation have a particle size of greater than 250 micrometers. In certain embodiments, at least 20% of particles of said formulation have a particle size of greater than 500 micrometers; at least 50% of particles of said formulation have a particle size of greater than 500 micrometers.

Embodiments can include one or more of the following features.

From 10 percent to 60 percent of the formulation have a particle size of less than 250 micrometers. From 10 percent to 30 percent of the formulation have a particle size of less than 250 micrometers.

From 20 percent to 90 percent of the formulation have a particle size that is greater than or equal to 250 micrometers. From 30 percent to 80 percent of the formulation have a particle size that is greater than or equal to 500 micrometers.

From 40 percent to 90 percent of the formulation have a particle size that is greater than or equal to 250 micrometers. From 40 percent to 80 percent of the formulation have a particle size that is greater than or equal to 500 micrometers.

From 10 percent to 60 percent of the formulation have a particle size of less than 250 micrometers; and from 40 percent to 90 percent of the formulation have a particle size that is greater than or equal to 250 micrometers. From 40 percent to 80 percent of the formulation have a particle size that is greater than or equal to 500 micrometers.

The formulation can have a particle size of at most about 1000 micrometers.

From 20 percent to 90 percent of the formulation have a particle size of from 250 micrometers to 1000 micrometers. From 30 percent to 70 percent of the formulation have a particle size of from 500 micrometers to 1000 micrometers.

From 40 percent to 90 percent of the formulation have a particle size of from 250 micrometers to 1000 micrometers. From 40 percent to 80 percent of the formulation have a particle size of from 500 micrometers to 1000 micrometers. From 40 percent to 80 percent of the formulation have a particle size of from 500 micrometers to 850 micrometers.

From 10 percent to 60 percent of the formulation can have a particle size of less than 250 micrometers; and from 40 percent to 90 percent of the formulation have a particle size of from 250 micrometers to 1000 micrometers. From 40 percent to 80 percent of the formulation have a particle size of from 500 micrometers to 1000 micrometers. From 40 percent to 80 percent of the formulation have a particle size of from 500 micrometers to 850 micrometers.

The compound of formula (I) (or salt thereof) is less than 20% crystalline. The compound of formula (I) (or salt thereof) is less than 10% crystalline. The compound of formula (I) (or salt thereof) is less than 5% crystalline.

When the formulation is stirred at 37° C. in a dissolution media selected from 0.1 N aqueous HCl and 0.1 N aqueous HCl/0.5% Tween and at an maximum concentration selected from 0.011 mg of the compound of formula (I)/mL of dissolution media, 0.033 mg of the compound of formula (I)/mL of dissolution media, and 0.133 mg of the compound of formula (I)/mL of dissolution media, dissolution of the compound of formula (I) is at least 75% complete after 90 minutes as determined by HPLC.

The formulation is stable upon actual or simulated storage at 5° C. for at least 6 months.

The formulation is stable upon actual or simulated storage at 25° C./60% relative humidity for at least 3 months.

The formulation is stable upon actual or simulated storage at 40° C./75% relative humidity for 1 month.

Administration of a single dose of the formulation to a beagle dog produces a mean peak plasma concentration (Cmax) of the compound of formula (I) of between 180 and 220 ng/mL for a formulation containing 30 mg of the compound of formula (I) and between 60 and 80 ng/mL for a formulation containing 10 mg of the compound of formula (I).

Daily administration of the formulation containing 30 mg of active compound of formula (I) to a beagle dog produces a mean steady state area under the concentration time curve (AUC_{(0-24 hrs)}) of the compound of formula (I) of between 7000 and 10,000 nghr/mL.

The formulations described herein include the compound of formula (I), or a pharmaceutically acceptable salt thereof (e.g., IPI-926), as the active ingredient. In some implementations of the subject matter described herein, the formulations include a pharmaceutically acceptable salt of the compound of formula (I), e.g., IPI-926. Such salts thus include both the compound of formula (I) itself, which is the biologically active moiety, and the accompanying salt-forming elements (e.g., H and Cl in the case of a hydrochloride salt). As such, the skilled artisan will appreciate that a given amount (e.g., mass or weight percent) of salt does not correspond to the same amount of biologically active moiety (i.e., the compound of formula (I) itself). For example, a 10 mg sample of a salt does not equate to 10 mg of the formula (I) compound itself Again, this is because the formula (I) compound itself is “diluted” by the presence of the salt forming elements and thus only constitutes a percentage of what is present in the total salt. To further illustrate, in some implementations of the subject matter described herein, the formula (I) compound is provided in the form of an HCl salt/IPA solvate. Here, the percent active moiety can be calculated by the following equation:

(100%−Percent impurities by HPLC−Percent Water Content by Karl Fisher−Percent residual solvents by GC)*(HCl salt correction);

in which the HCl Salt correction=1−((541.23−504.77)/541.23).

Thus, for the avoidance of doubt, the phrase “the active compound of formula (I)” as used herein is intended to refer only to the compound of formula (I) itself (sometimes referred to in terms of its total constituent atoms and connectivity as the free base form), i.e.:

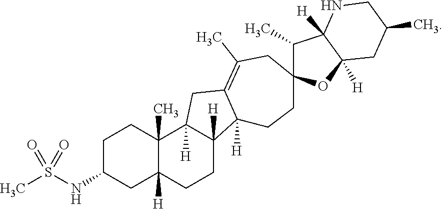

Reference, for example to a particular amount of the active compound of formula (I) in the formulations described herein refers only to the amount that is due to the compound of formula (I) itself (i.e., the active moiety).

In embodiments, the formulation includes between 5% and 50% (w/w) of the active compound of formula (I). In some embodiments, the formulation includes between 10% and 40% (w/w) of the active compound of formula (I). The formulation comprises between 20% and 30% (w/w) of the active compound of formula (I). In some embodiments, the formulation includes between 5% and 15% (w/w) of the active compound of formula (I). In some embodiments, the formulation includes from 5 milligrams to 500 milligrams of the active compound of formula (I). The formulation comprises 10 milligrams or 30 milligrams of the active compound of formula (I).

The formulation comprises from 110 milligrams to 130 milligrams of the active compound of formula (I). The formulation comprises 120 milligrams of the active compound of formula (I).

In certain embodiments, the compound is the hydrochloride salt (i.e., IPI-926).

In certain embodiments, the formulation is orally administered in a solid dosage form. In certain embodiments, the solid dosage form is a capsule or tablet (e.g., a capsule). In certain embodiments, the capsule is a gelatin capsule or a hydroxypropyl methylcellulose capsule.

In certain embodiments, the formulation further comprises a filler. In certain embodiments, the filler is selected from microcrystalline cellulose, lactose, compressible sugar, pregelatinized starch, dibasic calcium phosphate, tribasic calcium phosphate, and calcium sulfate. In certain embodiments, the filler is microcrystalline cellulose.

In certain embodiments, the formulation further comprises a binder. In certain embodiments, the binder is selected from polyvinylpyrrolidone, hydroxypropyl cellulose, methylcellulose, hydroxypropyl methylcellulose, pregelatizined starch, sucrose, and acacia gum. In certain embodiments, the binder is from polyvinylpyrrolidone.

In certain embodiments, the formulation further comprises a surfactant. In certain embodiments, the surfactant is selected from Tween 20, Tween 80, sodium laurel sulfate and sodium dodecyl sulfate. In certain embodiments, the surfactant is Tween 80.

In certain embodiments, the formulation further comprises a disintegrant. In certain embodiments, the disintegrant is selected from croscarmellose sodium, sodium starch glycolate, crospovidone, and starch.

In certain embodiments, the formulation is prepared by granulation.

Also provided are methods of making the above described formulations and methods of treating cancer comprising administering the above described formulation, alone or in combination with one or more additional cancer therapeutic agents.

In one aspect, methods of making a pharmaceutical formulation are featured, which include granulating a mixture of a compound of formula (I):

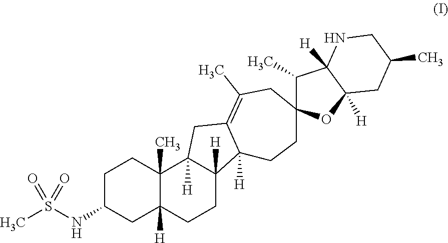

or a pharmaceutically acceptable salt thereof, and a liquid.

Embodiments can include one or more of the following features.

The liquid includes water. For example, the liquid can be an aqueous solution of a surfactant.

The ratio of the weight of the liquid to the weight of the total solid (i.e., compound of formula (I), or a pharmaceutically acceptable salt thereof, and if present, one or more additional pharmaceutically acceptable solids (e.g., one or more pharmaceutically-acceptable filler(s), binder(s), surfactant(s). and disintegrant(s); as well as one or more other therapeutic agent(s)) is greater than 0.25 (e.g., from 0.25 to 1.5; from 0.25 to 1; from 0.25 to 0.8; from 0.25 to 0.6; from 0.4 to 0.6; from 0.5 to 0.6; less than 1). In certain embodiments, from 25 weight percent to 80 percent, or from 50 weight percent to 80 weight percent (e.g., from 50 weight percent to 70 weight percent, from 55 weight percent to 70 weight percent, or from 60 weight percent to 70 weight percent) of water is used. For example, 50, 55, 57, 60, 62, 64, 65, 67, 68, 70, 72, 74, 75, 76, 78, 80 weight percent water can be used. In some implementations of the subject matter disecribed herein, 57.3 weight percent water is used. In other implementations of the subject matter disecribed herein, 64.5 weight percent water is used.

The method can further include granulating a filler in the mixture. The method can further include granulating a binder in the mixture. The method can further include the step of drying the granulation.

### BRIEF DESCRIPTION OF THE DRAWINGS

FIG. 1 is a graph depicting the percent release of IPI-926 of a direct blend of IPI-926 in Avicel™ PH-200 (40/60) in gelatin capsule and three granulated formulations in simulated gastrointestinal fluid (SGF) at pH 1.2 without enzymes over time: (i) a granulation formulation of IPI-926 blended Avicel™ PH-200 in gelatin capsule (water granulated); (ii) a granulation formulation of IPI-926+methylcellulose with blended Avicel™ PH-200 in gelatin capsule (MC granulated); and (iii) a granulation formulation of IPI-926+Tween-80+methylcellulose with blended Avicel™ PH-200 in gelatin capsule (Tween/MC granulated).

FIG. 2 is a graph depicting the exposure of IPI-926 in Beagle dogs after administration of a direct blend of IPI-926 in Avicel™ PH-200 (40/60) in gelatin capsule.

FIG. 3 is a graph depicting the exposure of IPI-926 in Beagle dogs upon administration of a suspension of IPI-926 in methylcellulose, Tween 80 and water and three encapsulated formulations: (i) a direct blend of IPI-926 and Avicel™ PH-200 (40/60) in gelatin capsule (direct blend); (ii) a granulation formulation of IPI-926+PVP with blended Avicel™ PH-200 in gelatin capsule (PVP granulated); and (iii) a granulation formulation of IPI-926+methylcellulose with blended Avicel™ PH-200 in gelatin capsule (MC granulated).

FIG. 4 is a graph depicting the percent release of IPI-926 from two granulation formulations in simulated gastrointestinal fluid (SGF) at pH 1.2 without enzymes over time: (i) IPI-926+PVP granulation formulation with blended Avicel™ PH-200 in gelatin capsule (PVP granulated); and (ii) IPI-926+methylcellulose granulation formulation with blended Avicel™ PH-200 in gelatin capsule (MC granulated).

FIG. 5 is a graph depicting the exposure of IPI-926 in Beagle dogs after administration of the PVP granulated formulation of FIG. 4 at 4 mg/kg and 8 mg/kg.

FIG. 6 is a graph depicting the percent release of modified PVP granulated formulations according to Table 2 at 10 mg, 30 mg and 120 mg in simulated gastrointestinal fluid (SGF) at pH 1.2 without enzymes over time.

FIG. 7 is a graph depicting the exposure of IPI-926 in Beagle dogs upon administration of 30 mg of IPI-926 in a suspension of methylcellulose, Tween 80 and water to two encapsulated formulations: (i) a 10 mg granulation formulation of IPI-926+PVP+Avicel™ PH-200+Tween 80 in HPMC capsule (10 mg capsule); and (ii) a 30 mg granulation formulation of IPI-926+PVP+Avicel™ PH-200+Tween 80 with blended Avicel™ PH-200 in HPMC capsule (30 mg capsule).

FIG. 8 is a graph depicting the exposure of IPI-926 in Beagle dogs upon administration of a single 60 mg capsule of the high potency formulation of Table 3 to two 30 mg capsules of the low potency formulation of Table 2.

FIG. 9 is a graph showing the release of IPI-926 over time in low potency formulations spiked with additional fines.

### DETAILED DESCRIPTION

This application features pharmaceutical formulations (e.g., solid dosage forms) that are useful for the oral administration of a compound of formula (I) (shown below), or a pharmaceutically acceptable salt thereof (e.g., IPI-926), to a human or animal subject.

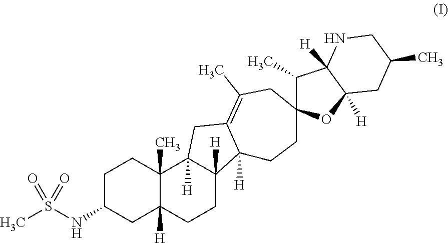

As used herein, the term “pharmaceutically acceptable salt” or “salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences (1977) 66:1-19. Pharmaceutically acceptable salts of the compounds of this invention include those derived from suitable inorganic and organic acids. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

In certain embodiments, the compound of formula (I) is a pharmaceutically acceptable salt.

In certain embodiments, the compound of formula (I) is the hydrochloride salt.

In some embodiments, the formulations include fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers).

In certain embodiments, not more than 80% (e.g., not more than 75%, not more than 70%, not more than 65%, not more than 60%, not more than 55%, not more than 50%, not more than 40%, not more than 30%, not more than 20%, not more than 10%) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers). In certain embodiments, not more than 80% of the formulation by weight have a particle size of less than 250 micrometers. In certain embodiments, not more than 80% of the formulation by weight have a particle size of less than 150 micrometers. In certain embodiments, not more than 60% of the formulation by weight have a particle size of less than 125 micrometers.

In certain embodiments, from 1 percent to 80 percent (e.g., from 1 percent to 75 percent, from 1 percent to 70 percent, from 1 percent to 65 percent, from 1 percent to 60 percent, from 1 percent to 55 percent, from 1 percent to 50 percent, from 1 percent to 45 percent, from 1 percent to 40 percent, from 1 percent to 35 percent, from 1 percent to 30 percent, from 1 percent to 20 percent, from 1 percent to 10 percent, from 1 percent to 5 percent) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers).

In certain embodiments, from 5 percent to 80 percent (e.g., from 5 percent to 75 percent, from 5 percent to 70 percent, from 5 percent to 65 percent, from 5 percent to 60 percent, from 5 percent to 55 percent, from 5 percent to 50 percent, from 5 percent to 45 percent, from 5 percent to 40 percent, from 5 percent to 35 percent, from 5 percent to 30 percent, from 5 percent to 20 percent, from 5 percent to 10 percent) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers).

In certain embodiments, from 10 percent to 80 percent (e.g., from 10 percent to 75 percent, from 10 percent to 70 percent, from 10 percent to 65 percent, from 10 percent to 60 percent, from 10 percent to 55 percent, from 10 percent to 50 percent, from 10 percent to 45 percent, from 10 percent to 40 percent, from 10 percent to 35 percent, from 10 percent to 30 percent, from 10 percent to 20 percent,) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers).

In certain embodiments, from 10 percent to 60 percent of the formulation by weight have a particle size of less than 250 micrometers. In certain embodiments, from 10 percent to 30 percent of the formulation by weight have a particle size of less than 250 micrometers.

In some embodiments, from 20 percent to 99 percent (e.g., from 20 percent to 95 percent, from 20 percent to 90 percent, from 20 percent to 85 percent, from 20 percent to 80 percent, from 20 percent to 75 percent, from 20 percent to 70 percent, from 20 percent to 65 percent, from 20 percent to 60 percent, from 20 percent to 55 percent, from 20 percent to 50 percent, from 20 percent to 45 percent, from 20 percent to 40 percent, from 20 percent to 35 percent, from 20 percent to 30 percent) of the formulation by weight have a particle size that is greater than or equal to 250 micrometers. In certain embodiments, from 20 percent to 90 percent of the formulation by weight have a particle size that is greater than or equal to 250 micrometers. In certain embodiments, at least or more than 20 percent (e.g., at least or more than 30 percent, at least or more than 40 percent, at least or more than 50 percent, at least or more than 60 percent, at least or more than 70%, at least or more than 80 percent, at least or more than 90%) of the formulation by weight have a particle size that is greater than or equal to 250 micrometers (e.g., greater than 250 micrometers). In certain embodiments, at least or more than 20 percent (e.g., at least or more than 30 percent, at least or more than 40 percent, at least or more than 50 percent, at least or more than 60 percent, at least or more than 70%, at least or more than 80 percent, at least or more than 90%) of the formulation by weight have a particle size that is greater than or equal to 500 micrometers (e.g., greater than 500 micrometers).

In certain embodiments, from 30 percent to 80 percent (e.g., from 30 percent to 75 percent, from 30 percent to 70 percent, from 30 percent to 65 percent, from 30 percent to 60 percent, from 30 percent to 55 percent, from 30 percent to 50 percent, from 30 percent to 45 percent, from 30 percent to 40 percent) of the formulation by weight have a particle size that is greater than or equal to 500 micrometers.

In some embodiments, from 40 percent to 99 percent (e.g., from 40 percent to 95 percent, from 40 percent to 90 percent, from 40 percent to 85 percent, from 40 percent to 80 percent, from 40 percent to 75 percent, from 40 percent to 70 percent, from 40 percent to 65 percent, from 40 percent to 60 percent, from 40 percent to 55 percent, from 40 percent to 50 percent) of the formulation by weight have a particle size that is greater than or equal to 250 micrometers. In certain embodiments, from 40 percent to 90 percent of the formulation by weight have a particle size that is greater than or equal to 250 micrometers. In certain embodiments, at least or more than 50 percent (e.g., at least or more than 60 percent, at least or more than 70%, at least or more than 80 percent, at least or more than 90%) of the formulation by weight have a particle size that is greater than or equal to 250 micrometers (e.g., greater than 250 micrometers). In certain embodiments, at least or more than 50 percent (e.g., at least or more than 60 percent, at least or more than 70%, at least or more than 80 percent, at least or more than 90%) of the formulation by weight have a particle size that is greater than or equal to 500 micrometers (e.g., greater than 500 micrometers).

In certain embodiments, from 40 percent to 80 percent (e.g., from 40 percent to 75 percent, from 40 percent to 70 percent, from 40 percent to 65 percent, from 40 percent to 60 percent, from 40 percent to 55 percent, from 40 percent to 50 percent) of the formulation by weight have a particle size that is greater than or equal to 500 micrometers. In certain embodiments, at least or more than 50 percent (e.g., at least or more than 60 percent, at least or more than 70%) of the formulation by weight have a particle size that is greater than or equal to 500 micrometers (e.g., greater than 500 micrometers).

In some embodiments: (i) from 1 percent to 80 percent (e.g., from 5 percent to 80 percent, from 10 percent to 80 percent, and including any sub-ranges described herein) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers); and (ii) from 20 percent to 99 percent (e.g., from 20 percent to 90 percent, from 40 percent to 90 percent, and including any sub-ranges described herein) of the formulation by weight have a particle size that is greater than or equal to 250 micrometers. In embodiments, from 30 percent to 80 percent (e.g., from 40 percent to 80 percent, and including any sub-ranges described herein) of the formulation by weight have a particle size that is greater than or equal to 500 micrometers.

In certain embodiments, from 10 percent to 60 percent of the formulation by weight have a particle size of less than 250 micrometers; and from 40 percent to 90 percent of the formulation by weight have a particle size that is greater than or equal to 250 micrometers. In embodiments, from 40 percent to 80 percent of the formulation by weight have a particle size that is greater than or equal to 500 micrometers.

In some embodiments, wherein the formulation has a particle size of at most about 1000 micrometers.

In certain embodiments, from 20 percent to 99 percent (e.g., from 20 percent to 95 percent, from 20 percent to 90 percent, from 20 percent to 85 percent, from 20 percent to 80 percent, from 20 percent to 75 percent, from 20 percent to 70 percent, from 20 percent to 65 percent, from 20 percent to 60 percent, from 20 percent to 55 percent, from 20 percent to 50 percent, from 20 percent to 45 percent, from 20 percent to 40 percent, from 20 percent to 35 percent, from 20 percent to 30 percent) of the formulation by weight have a particle size that is from 250 micrometers to 1000 micrometers. In certain embodiments, from 20 percent to 90 percent of the formulation by weight have a particle size that is from 250 micrometers to 1000 micrometers. In certain embodiments, at least or more than 20 percent (e.g., at least or more than 30 percent, at least or more than 40 percent, at least or more than 50 percent, at least or more than 60 percent, at least or more than 70%, at least or more than 80 percent, at least or more than 90%) of the formulation by weight have a particle size that is from greater than or equal to 250 micrometers to 1000 micrometers (e.g., greater than 250 micrometers to 1000 micrometers). In certain embodiments, at least or more than 20 percent (e.g., at least or more than 30 percent, at least or more than 40 percent, at least or more than 50 percent, at least or more than 60 percent, at least or more than 70%, at least or more than 80 percent, at least or more than 90%) of the formulation by weight have a particle size that is greater than or equal to 500 micrometers to 1000 micrometers (e.g., greater than 500 micrometers to 1000 micrometers).

In certain embodiments, from 30 percent to 80 percent (e.g., from 30 percent to 75 percent, from 30 percent to 70 percent, from 30 percent to 65 percent, from 30 percent to 60 percent, from 30 percent to 55 percent, from 30 percent to 50 percent, from 30 percent to 45 percent, from 30 percent to 40 percent) of the formulation by weight have a particle size that is 500 micrometers to 1000 micrometers.

In some embodiments, from 40 percent to 99 percent (e.g., from 40 percent to 95 percent, from 40 percent to 90 percent, from 40 percent to 85 percent, from 40 percent to 80 percent, from 40 percent to 75 percent, from 40 percent to 70 percent, from 40 percent to 65 percent, from 40 percent to 60 percent, from 40 percent to 55 percent, from 40 percent to 50 percent) of the formulation by weight have a particle size that is greater than or equal to 250 micrometers. In certain embodiments, from 40 percent to 90 percent of the formulation by weight have a particle size that is from 250 micrometers to 1000 micrometers. In certain embodiments, more than 50 percent of the formulation by weight have a particle size that is from 250 micrometers to 1000 micrometers. In certain embodiments, at least or more than 50 percent (e.g., at least or more than 60 percent, at least or more than 70%, at least or more than 80 percent, at least or more than 90%) of the formulation by weight have a particle size that is from 500 micrometers to 1000 micrometers (e.g., >500 micrometers to 1000 micrometers).

In certain embodiments, from 40 percent to 80 percent (e.g., from 40 percent to 75 percent, from 40 percent to 70 percent, from 40 percent to 65 percent, from 40 percent to 60 percent, from 40 percent to 55 percent, from 40 percent to 50 percent) of the formulation by weight have a particle size that is from 500 micrometers to 1000 micrometers (e.g., from 500 micrometers to 850 micrometers). In certain embodiments, more than 50 percent (e.g., more than 60 percent, more than 70%) of the formulation by weight have a particle size that is from 500 micrometers to 1000 micrometers (e.g., >500 micrometers to 1000 micrometers).

In some embodiments: (i) from 1 percent to 80 percent (e.g., from 5 percent to 80 percent, from 10 percent to 80 percent, and including any sub-ranges described herein) of the formulation by weight are fines (e.g., having a particle size of less than 250 micrometers, less than 150 micrometers, less than 125 micrometers, from 250 micrometers to 150 micrometers, from 150 micrometers to 125 micrometers, or any combination thereof; e.g., less than 250 micrometers); and (ii) from 20 percent to 99 percent (e.g., from 20 percent to 90 percent, from 40 percent to 90 percent, and including any sub-ranges described herein) of the formulation by weight have a particle size that is from 250 micrometers to 1000 micrometers. In embodiments, from 30 percent to 80 percent (e.g., from 40 percent to 80 percent, and including any sub-ranges described herein) of the formulation by weight have a particle size that is from 500 micrometers to 1000 micrometers (e.g., from 500 micrometers to 850 micrometers).

In certain embodiments, from 10 percent to 60 percent of the compound of the formulation by weight have a particle size of less than 250 micrometers; and from 40 percent to 90 percent of the formulation by weight have a particle size that is from 250 micrometers to 1000 micrometers. In embodiments, from 40 percent to 80 percent of the formulation by weight have a particle size that is from 500 micrometers to 1000 micrometers (e.g., from 500 micrometers to 850 micrometers).

In certain embodiments, the formulation further includes a pharmaceutically acceptable excipient (which include the pharmaceutically acceptable solids described herein that can be present in the solid dosage forms described herein).

Pharmaceutically acceptable excipients include any and all fillers, binders, surfactants, disintegrants, sugars, polymers, antioxidants, solubilizing or suspending agents, chelating agents, preservatives, buffering agents and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various pharmaceutically acceptable excipients used in preparing pharmaceutically acceptable formulations and known techniques for the preparation thereof. Except insofar as any conventional carrier medium is incompatible with the formulations disclosed herein, such as by producing any undesirable biological effect or otherwise interacting in a deleterious manner with any component of the formulation, its use is contemplated to be within the scope of this invention.

For example, in certain embodiments, the formulation further includes a filler.

Fillers include, but are not limited to, calcium carbonate, sodium carbonate, calcium phosphate, dibasic calcium phosphate, tribasic calcium phosphate, calcium sulfate, calcium hydrogen phosphate, sodium phosphate lactose, compressible sugar (e.g., powdered sugar), microcrystalline cellulose (e.g., Avicel™ PH-101, Avicel™ PH-102, Avicel™ PH-103, Avicel™ PH-105 and Avicel™ PH-200), a coprocessed mixture of lactose and pulverized cellulose (Cellactose™), kaolin, mannitol, sorbitol, inositol, sodium chloride and pregelatinized starch.

In certain embodiments, the filler is selected from microcrystalline cellulose, lactose, compressible sugar, pregelatinized starch, dibasic calcium phosphate, tribasic calcium phosphate, and calcium sulfate.

In certain embodiments, the filler is microcrystalline cellulose.

In certain embodiments, when the formulation includes a salt form of the compound of formula (I), the amount of filler (e.g., microcrystalline cellulose, e.g., Avicel PH200) is reduced to accommodate the extra mass associated with the salt-forming elements. For example, when manufacturing IPI-926 drug product, the weights of IPI-926 and Avicel PH 200 intragranular are adjusted using the following calculation: Adjusted weight of IPI-926=(Theoretical amount IPI-926/(IPI-926% active moiety/100)). Adjusted weight of Avicel PH200 (intragranular)=(Theoretical amount of IPI-926+Theoretical amount of Avicel PH 200 intragranular)−Adjusted weight of IPI-926.

In certain embodiments, the formulation includes a binder.

Binders include, but are not limited to, starch (e.g. cornstarch and starch paste); gelatin; sugars (e.g. sucrose, glucose, dextrose, dextrin, molasses, lactose, lactitol, mannitol, etc.); natural and synthetic gums (e.g. acacia gum, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone, magnesium aluminum silicate (Veegum), and larch arabogalactan); alginates; polyethylene oxide; polyethylene glycol; inorganic calcium salts; silicic acid; polymethacrylates and waxes.

In certain embodiments, the binder is selected from polyvinylpyrrolidone, hydroxypropyl cellulose, methylcellulose, hydroxypropyl methylcellulose, pregelatizined starch, sucrose, and acacia gum.

In certain embodiments, the binder is polyvinylpyrrolidone (PVP). Polyvinylpyrrolidones are nonionic water-soluble polymers and include vinylpyrrolidone homopolymers and copolymers with different molecular weights. Differentiations among PVP's of different molecular weight are typically made on the basis of the PVP's K-value, which represents a viscosity index relating to molecular weight of the PVP. The K-value can be calculated using Fikentscher's formula and the relative viscosity of aqueous polyvinylpyrrolidone solution to water, the latter being measured by capillary viscometer at 25° C. Examples of PVP grades based on K-value (indicated with parentheses) include 12 (11-14); 17 (16-18); 25 (24-27); 30 (28-32); and 90 (85-95) (see, e.g., www.springerlink.com). In some implementations of the subject matter described herein, the binder is PVP-30.

In certain embodiments, the formulation further includes a surfactant.

Surfactants include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), Tween surfactants (e.g., sorbitan fatty acid esters such as polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate

[Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate (SLS), sodium dodecyl sulfate (SDS), Pluronic F 68, Poloxamer 188, cetrimonium bromide, cetylpyridinium chloride, benzalkonium chloride and docusate sodium.

In certain embodiments, the surfactant is selected from a Tween surfactant, sodium laurel sulfate and sodium dodecyl sulfate. In certain embodiments, the surfactant is a Tween surfactant. In certain embodiments, the surfactant is Tween 80.

In certain embodiments, the formulation further includes a disintegrant.

Disintegrants include, but are not limited to, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), calcium carboxymethyl cellulose, carboxymethyl cellulose, cross-linked sodium carboxymethyl cellulose (croscarmellose sodium; AcDiSol™), methylcellulose, sodium carboxymethyl starch (sodium starch glycolate), starch (e.g., potato starch, corn starch, tapioca starch, pregelatinized starch (starch 1500), microcrystalline starch, water insoluble starch), magnesium aluminum silicate (Veegum) and sodium lauryl sulfate (SLS).

In certain embodiments, the disintegrant is selected from croscarmellose sodium, sodium starch glycolate, crospovidone, and starch. In certain embodiments, the disintegrant is croscarmellose sodium (AcDiSol™).

In certain embodiments, the formulation includes a compound of formula (I) or salt thereof, a filler selected from Avicel™ PH-200, a binder selected from polyvinylpyrrolidine (PVP) (e.g., PVP-30) and a surfactant selected from Tween 80. In certain embodiments, the formulation further includes a disintegrant selected from croscarmellose sodium (AcDiSol™).

Other excipients which may further be provided as components of the pharmaceutical composition includes various sugars, polymers, antioxidants, solubilizing or suspending agents, chelating agents, preservatives, buffering agents and/or lubricating agents.

Sugars include, but are not limited to, glycerol, polyvinylalcohol, propylene glycol, sorbitol, ribose, arabinose, xylose, lyxose, allose, altrose, mannose, mannitol, gulose, dextrose, idose, galactose, talose, glucose, fructose, dextrates, lactose, sucrose, starches (i.e., amylase and amylopectin), sodium starch glycolate, cellulose and cellulose derivatives (i.e., methylcellulose, hydroxypropyl cellulose, hydroxyethyl cellulose, hydroxyethylmethyl cellulose, carboxymethyl cellulose, cellulose acetate, cellulose acetate phthalate, croscarmellose, hypomellose, and hydroxypropyl methyl cellulose), carrageenan, cyclodextrins (e.g., hydroxypropyl-gamma-CD), dextrin, polydextrose, and trehalose.

Polymers include, but are not limited to, polyvinyl alcohol (PVA), gelatin, polyvinyl pyrolidone (PVP), albumin, polyethyleneimine (PEI), acacia gum, cellulose derivatives, calcium polypectate, maleic anhydride derivatives, polyacrylic and methacrylic acid, phospholipids, glycols (such as propylene glycol or polyethylene glycol), polyglycolide and lactide derivatives, polyethylene-polyoxypropylene-block polymers, starch, waxes, oils, alginates and alginic acid, calcium caseinate, carrageenan, pectins, polyhexametaphosphate, polyvinyl acetate and polyvinyl alcohol.

Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, sodium sulfite, cysteine hydrochloride, thioglycerol, sodium mercaptoacetate, sodium formaldehyde sulfoxylate (SFS), lecithin and organic phosphites (e.g., dimethyl phosphite, diethyl phosphite, dibutyl phosphite, triethyl phosphite, tris(2-chloroethyl)phosphite, and tris(2-4-t-butyl-phenyl)-phosphite, etc.).

Solubilizing or suspending agents include, but are not limited to, water, organic solvents and oils such as almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, butyl stearate, caprylic triglyceride, capric triglyceride, cyclomethicone, diethyl sebacate, dimethicone 360, isopropyl myristate, mineral oil, and silicone oil.

Chelating agents include, but are not limited to, ethylenediaminetetraacetic acid (EDTA), citric acid monohydrate, disodium edetate, dipotassium edetate, edetic acid, fumaric acid, malic acid, phosphoric acid, sodium edetate, tartaric acid and trisodium edetate.

Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol and thimerosal.

Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate and sorbic acid.

Alcohol preservatives include, but are not limited to, ethanol, polyethylene glycol, phenol, phenolic compounds, bisphenol, chlorobutanol, hydroxybenzoate and phenylethyl alcohol.

Acidic preservatives include, but are not limited to, vitamin A, vitamin C, vitamin E, beta-carotene, citric acid, acetic acid, dehydroacetic acid, ascorbic acid, sorbic acid and phytic acid.

Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon and Euxyl.

Buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, and ethyl alcohol.

Lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, and sodium lauryl sulfate.

In some embodiments, the one or more pharmaceutically acceptable excipients added to the pharmaceutical composition are at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Dosage and Administration

Although the descriptions of the formulations provided herein are principally suitable for administration to humans, it will be understood by the skilled artisan that such compositions are generally suitable for administration to animals of all sorts (e.g., primates, cattle, pigs, horses, sheep, cats, dogs, and birds). Modification of formulations suitable for administration to humans in order to render the formulations suitable for administration to various animals is well understood, and the ordinarily skilled veterinary pharmacologist can design and/or perform such modification with merely ordinary, if any, experimentation.

Relative amounts of the compound of formula (I) or salt thereof and pharmaceutically acceptable excipients in a pharmaceutically acceptable formulation described herein will vary, depending upon the identity, size, and/or condition of the subject treated and further depending upon the route by which the formulation is to be administered. An effective amount of the active compound of formula (I) will vary from subject to subject, depending on species, age, and general condition of a subject, severity of the side effects or disorder, identity of the particular compound(s), mode of administration, and the like. As used herein, “an effective amount” refers to an amount of the active compound of formula (I) that confers a therapeutic effect (e.g., controls, relieves, ameliorates, alleviates, or slows the progression of); or prevents (e.g., delays the onset of or reduces the risk of developing) a disease, disorder, or condition or symptoms thereof on the treated subject. The therapeutic effect may be objective (i.e., measurable by some test or marker) or subjective (i.e., subject gives an indication of or feels an effect).

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). As will be appreciated by the skilled artisan, the desired doage can also be achieved by administration of two or more of the same or different dosage forms. This application describes a variety of dosage forms (e.g., capsules) containing differing amounts of the active compound of formula (I). The amount of the active compound of formula (I) present in said dosage forms is, however, not intended to explicitly or implicitly imply any limitation on the amount of the active compound of formula (I) that can be administered at any one or more times of administration (e.g., the amount present in any of the dosage forms described herein is not intended to explicitly or implicitly imply that the amount present represents, e.g., a maximum tolerated dose). Rather, should the desired dose of the active compound of formula (I) be greater than an amount present in any of the dosage forms described herein, then the skilled artisan will recognize that the desired dosage can be achieved by administration of two or more of the same or different dosage forms (e.g., a desired dosage of 150 mg of the active compound of formula (I) can be achieved by administration of a capsule containing 120 mg of the active compound of formula (I) and another capsule containing 30 mg of the active compound of formula (I).

In certain embodiments, the formulations may include from 0.1% weight percent to 100 weight percent of the compound of formula (I) or salt thereof delivered once a day. For ease of exposition, expressions such as “from 0.1% weight percent to 100 weight percent of the compound of formula (I)” are sometimes expressed herein as “between 0.1% and 100% (w/w) of the compound of formula (I) or salt thereof.” In certain embodiments, the formulations may include between 0.1% and 80% (w/w) of the compound of formula (I) or salt thereof delivered once a day.

In certain embodiments, the formulations may include between 0.1% and 100% (w/w) of the active compound of formula (I) delivered once a day. In certain embodiments, the formulations may include between 0.1% and 80% (w/w) of the active compound of formula (I) delivered once a day.

In certain embodiments, the formulations include between 1% and 80% (w/w), between 1% and 70% (w/w), between 1% and 60% (w/w), between 1% and 50% (w/w), between 1% and 40% (w/w), between 1% and 30% (w/w), between 1% and 20% (w/w), between 1% and 15% (w/w), between 1% and 10% (w/w) of a compound of formula (I) or salt thereof. In certain embodiments, the formulations include between 1% and 80% (w/w), between 1% and 70% (w/w), between 1% and 60% (w/w), between 1% and 50% (w/w), between 1% and 40% (w/w), between 1% and 30% (w/w), between 1% and 20% (w/w), between 1% and 15% (w/w), between 1% and 10% (w/w) of the active compound of formula (I).

In certain embodiments, the formulations include between 5% and 80% (w/w), between 5% and 70% (w/w), between 5% and 60% (w/w), between 5% and 50% (w/w), between 5% and 40% (w/w), between 5% and 30% (w/w), between 5% and 20% (w/w), between 5% and 15% (w/w), between 5% and 10% (w/w) of a compound of formula (I) or salt thereof.

In certain embodiments, the formulations include between 5% and 80% (w/w), between 5% and 70% (w/w), between 5% and 60% (w/w), between 5% and 50% (w/w), between 5% and 40% (w/w), between 5% and 30% (w/w), between 5% and 20% (w/w), between 5% and 15% (w/w), between 5% and 10% (w/w) of the active compound of formula (I).

In certain embodiments, the formulations include between 5% and 50% (w/w), between 5% and 45% (w/w), between 5% and 40% (w/w), between 5% and 35% (w/w), between 5% and 30% (w/w), between 5% and 25% (w/w), between 5% and 20% (w/w), between 5% and 15% (w/w), between 5% and 10% (w/w) of a compound of formula (I) or salt thereof.

In certain embodiments, the formulations include between between 5% and 50% (w/w), between 5% and 45% (w/w), 5% and 40% (w/w), between 5% and 35% (w/w), between 5% and 30% (w/w), between 5% and 25% (w/w), between 5% and 20% (w/w), between 5% and 15% (w/w), between 5% and 10% (w/w) of the active compound of formula (I).

In certain embodiments, the formulations include between 10% and 80% (w/w), between 10% and 70% (w/w), between 10% and 60% (w/w), between 10% and 50% (w/w), between 10% and 40% (w/w), between 10% and 30% (w/w), between 10% and 20% (w/w), between 10% and 15% (w/w) of a compound of formula (I) or salt thereof.

In certain embodiments, the formulations include between 10% and 80% (w/w), between 10% and 70% (w/w), between 10% and 60% (w/w), between 10% and 50% (w/w), between 10% and 40% (w/w), between 10% and 30% (w/w), between 10% and 20% (w/w), between 10% and 15% (w/w) of the active compound of formula (I).

In certain embodiments, the formulations include between 10% and 50% (w/w), between 10% and 40% (w/w), between 10% and 30% (w/w), between 10% and 20% (w/w), between 10% and 15% (w/w) of a compound of formula (I) or salt thereof.

In certain embodiments, the formulations include between 10% and 50% (w/w), between 10% and 40% (w/w), between 10% and 30% (w/w), between 10% and 20% (w/w), between 10% and 15% (w/w) of the active compound of formula (I).

In certain embodiments, the formulations include between 20% and 80% (w/w), between 20% and 60% (w/w), between 20% and 40% (w/w), between 30% and 80% (w/w), between 40% and 80% (w/w), between 50% and 80% (w/w) of a compound of formula (I) or salt thereof.

In certain embodiments, the formulations include between 20% and 80% (w/w), between 20% and 60% (w/w), between 20% and 40% (w/w), between 30% and 80% (w/w), between 40% and 80% (w/w), between 50% and 80% (w/w) of the active compound of formula (I).

In certain embodiments, an effective amount of the active compound of formula (I) for administration one or more times a day to a 70 kg adult human may include from 0.1 mg to 500 mg of per unit dosage form. For example, in certain embodiments, the effective daily dose can include from between 30 mg and 500 mg, or between 50 mg and 350 mg, or between 75 mg and 300 mg, or between about 100 mg and 250 mg, or between about 100 mg and 210 mg, or between about 110 mg and 170 mg of active compound of formula (I)/day. It will be appreciated that dose ranges as described herein provide guidance for the administration of provided pharmaceutically acceptable formulations to an adult. The amount to be administered to, for example, a child or an adolescent can be determined by a medical practitioner or person skilled in the art and can be lower or the same as that administered to an adult.

In certain embodiments, the formulations include between 0.1 mg and 500 mg; between 0.5 mg and 250 mg, between 1 mg and 200 mg; between 5 mg and 500 mg, between about 5 mg to 150 mg; or between 10 mg and 120 mg of the active compound of formula (I). (e.g., 5, 10, 20, 30, 40, 50, 60, 70, 80, 90, 100, 120, 125, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 mg).

In certain embodiments, the formulation includes at least 0.5 mg, at least 1 mg, at least 2 mg, at least 4 mg, at least 8 mg, at least 10 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 175 mg, at least 190 mg, at least 200 mg, at least 210 mg, or at least 220 mg of the active compound of formula (I).

In certain embodiments, the formulation includes about 4 mg, about 8 mg, 10 mg, 30 mg, or 120 mg of the active compound of formula (I).

In certain embodiments, the formulation includes from 5 weight percent to 50 weight percent of the compound of formula (I) or salt thereof. In certain embodiments, the formulation includes from 5 weight percent to 15 weight percent of the active compound of formula (I). Such formulations can also include from 5 milligrams to 40 milligrams of the active compound of formula (I), e.g., 10 milligrams or 30 milligrams of the active compound of formula (I). In other embodiments, the formulation includes from 20 weight percent to 30 weight percent of the active compound of formula (I). Such formulations can also include from 110 milligrams to 130 milligrams of the active compound of formula (I), e.g., 120 milligrams of the active compound of formula (I).

In certain embodiments, the composition comprises between 20% and 95% (w/w), between 30% and 95% (w/w), between 40% and 95% (w/w), between 40% and 90% (w/w), between 40% and 85% (w/w), between 40% and 80% (w/w), between 40% and 70% (w/w), between 50% and 85% (w/w) or between 60% and 85% (w/w) of a filler.

In certain embodiments, the composition comprises between about 0.1% and 50% (w/w), between 0.1% and 40% (w/w); between 0.1% and 30% (w/w); between 0.1% and 20% (w/w); between 0.1% and 10% (w/w); between 0.1% and 5% (w/w); between 1% and 5% (w/w); or between 2% and 5% (w/w) of a binder.

In certain embodiments, the composition comprises between 0.1% to 50% (w/w), between 0.1% and 40% (w/w); between 0.1% and 30% (w/w); between 0.1% and 20% (w/w); between 0.1% and 10% (w/w); between 1% and 10% (w/w); or between 2% and 10% (w/w) of a surfactant.

In certain embodiments, the formulation includes between 0.1% and 50% (w/w), between 0.1% and 40% (w/w); between 0.1% and 30% (w/w); between 1% and 30% (w/w); between 1% and 10%,); between 1% and 5%,between 5% and 30% (w/w); between 10% and 25% (w/w) of a disintegrant.

In certain embodiments, the formulation includes between 5% and 40% (w/w) of a compound of formula (I) or salt thereof, between 40% and 85% (w/w) of a filler, between 2% and 5% (w/w) of a binder, and between 2% and 10% (w/w) of a surfactant. In certain embodiments, the formulation further comprises between 10% and 25% (w/w) of a disintegrant.

Preparation

Provided herein are pharmaceutical formulations for oral administration. Such oral pharmaceutical formulations may be prepared by any method known or hereafter developed in the art of pharmacology (see, e.g., Remington's Pharmaceutical Sciences, Sixteenth Edition, E. W. Martin, Mack Publishing Co., Easton, Pa., 1980). In general, these methods include the step of bringing the compound of formula (I) or salt thereof into association with a pharmaceutically acceptable excipient and/or one or more other additional excipients, and then, if necessary and/or desirable, shaping and/or packaging the product into a desired single dosage form.

For example, in one aspect, provided herein are methods of making a pharmaceutical formulation which includes granulating a compound of formula (I) or a pharmaceutically acceptable salt thereof.

In certain embodiments, the granulating is dry granulation or wet granulation.

Granulation has been found to improve the flow of powder mixtures and mechanical properties of tablets. Wet granulation involves mixing a liquid with the drug product and, optionally, one or more excipients. Larger quantities of granulating liquid produce a narrower particle size range and coarser and harder granules (i.e., the proportion of fine granulate particles decreases). Wet granulation is used to improve flow, compressibility, bio-availability, homogeneity, electrostatic properties, and stability of solid dosage forms. Dry granulation, on the other hand, involves compressing a blend of the drug product and one or more excipients, followed by milling. After the milling, larger particles containing both drug product and excipient remain, similar to wet granulation.

For example, in one aspect, the present invention provides a process of making a pharmaceutical composition comprising granulating a mixture of a compound of formula (I) or a pharmaceutically acceptable salt thereof and a liquid.

Exemplary liquids include water or an aqueous solution of a pharmaceutically acceptable excipient. In certain embodiments, the liquid is an aqueous solution of a surfactant; for example, a Tween surfactant.

In certain embodiments, the method further comprises granulating a filler in the mixture. In certain embodiments, the method further comprises granulating a binder in the mixture.

In certain embodiments, provided is a method of making a pharmaceutical composition comprising granulating a mixture containing a compound of formula (I) or salt thereof, a filler and a binder with an aqueous solution of a surfactant.

In certain embodiments, the method further comprises the step of drying the granulation.

In certain embodiments, the method further comprises the step of blending an excipient with the dried granulation, for example, a filler or a disintegrant.

In certain embodiments, the method further comprises the step of screening the dried granulation.

In certain embodiments, the granulated formulation is further shaped and/or packaged into a single dosage form. In certain embodiments, the dosage form is a liquid dosage form. In certain embodiments, the dosage form is a solid dosage form.

Liquid dosage forms for oral administration include, but are not limited to, pharmaceutically acceptable emulsions, microemulsions, solutions, suspensions, syrups and elixirs.

Solid dosage forms for oral administration include tablets, dragees, capsules, pills and granules. In some implementations of the subject matter described herein, the solid dosage form for oral administration is a capsule.

In certain embodiments, the formulation is orally administered in a solid dosage form.

In certain embodiments, the solid dosage form is a capsule or tablet. In certain embodiments, the solid dosage form is a capsule. In certain embodiments, the capsule is a gelatin capsule or a hydroxypropyl methylcellulose (HPMC) capsule.

Methods of Treatment

Provided herein are methods of treating a proliferative disorder, such as cancer, comprising orally administering a formulation, as described above and herein, to a patient in need thereof.

A patient to which administration is contemplated includes, but is not limited to, humans (e.g., male, female, infant, child, adolescant, adult, elderly, etc.) and/or other primates; mammals, including commercially relevant mammals such as cattle, pigs, horses, sheep, cats, and/or dogs; and/or birds, including commercially relevant birds such as chickens, ducks, geese, and/or turkeys.

“Treating,” as used herein, refers to administering the minimal amount or concentration of a compound of formula (I) or salt thereof that, when administered, confers a therapeutic effect (e.g., controls, relieves, ameliorates, alleviates, or slows the progression of); or prevents (e.g., delays the onset of or reduces the risk of developing) a disease, disorder, or condition or symptoms thereof on the treated subject. In some implementations of the subject matter described herein, treating confers a therapeutic effect (e.g., controls, relieves, ameliorates, alleviates, or slows the progression of) a disease, disorder, or condition or symptoms thereof on the treated subject. In other implementations of the subject matter described herein, treating prevents (e.g., delays the onset of or reduces the risk of developing).

IPI-926, described in PCT publications WO 2008083252 and WO 2008083248, both of which are incorporated herein by reference, has been shown to inhibit in vitro growth of human cell lines derived from patients with pancreatic cancer, medulloblastoma, lung cancer, multiple myeloma, acute lymphocytic leukemia, myelodysplatic syndrome, non-Hodgkin's type lymphoma, Hodgkin's disease and lymphocygtic leukemia.

IPI-926 has also shown tumor growth inhibition in a number of preclinical in vivo models, such as medulloblastoma (Pink et al., “Activity of IPI-926, a potent HH pathway inhibitor, in a novel model of medulloblastoma derived from Ptch/HIC+/−mice” American Association for Cancer Research, 1588, 2008; Villavicencia et al., “Activity of the Hh pathway inhibitor IPI-926 in a mouse model of medulloblastoma” American Association for Cancer Research, 2009); small cell lung cancer (Travaglione et al., “A novel Hh pathway inhibitor, IPI-926, delays recurrence post-chemotherapy in a primary human SCLC xenograft model”, American Association for Cancer Research, 4611, 2008; Peacock et al., “Visualization of SMOOTHENED activation supports an essential role for Hedgehog signaling in the regulation of self-renewal in small cell lung cancer” American Association for Cancer Research, 2009); non-small cell lung cancer (Mandley, E., et al. The Hh inhibitor IPI-926 delays tumor re-growth of a non-small cell lung cancer xenograft model following treatment with an EGFR targeted tyrosine kinase inhibitor. American Association for Cancer Research, 2010), skin cancer, head and neck cancer, and ovarian cancer (Growdon et al, “Hedgehog pathway inhibitor cyclopamine suppresses Glil expression and inhibits serous ovarian cancer xenograft growth.” Society of Gynecologic Oncologists Annual Meeting on Women's Cancer, 2009).

Additionally, IPI-926 has demonstrated rapid and sustained Hedgehog pathway inhibition in stromal cells, a downstream mediator of Hedgehog signaling, after single administration in a model of human pancreatic cancer (Traviglione et al., “Activity of IPI-926, a novel inhibitor of the Hh pathway, in subcutaneous and orthotopically implanted xenograft tumors that express SHh ligand.” EORTC-NCI-AACR Symposium on “Molecular Targets and Cancer Therapeutics” 2008).

IPI-926 is also being investigated in clinical trials. Inhibition of the hedgehog pathway has also been shown to reduce or inhibit the growth of a variety of cancers, such as acute lymphocytic leukemia (ALL) (Ji et al., Journal of Biological Chemistry (2007) 282:37370-37377); acute myeloid leukemia (AML), basal cell carcinoma (Xie et al., Nature (1998) 391:90-92; Williams et al., PNAS (2003) 100:4616-4621; Bale and Yu (2001) Human Molecular Genetics (2001) 10:757-762); biliary cancer (Berman et al., Nature (2003) 425:846-851; WO 2005/013800); brain cancer and glioma (Clement et al., Current Biology (2007) 17:1-8; Ehtesham et al., Ongogene (2007) 1-10); bladder cancer; breast cancer (Kubo et al., Cancer Research (2004) 64:6071-6074; Lewis et al., J. Mammary Gland Biology and Neoplasia (2004) 2:165-181); chondrosarcoma (Wunder et al., Lancet Oncology (2007) 513-524); chronic lymphocytic leukemia (CLL) (Hedge et al., Mol. Cancer Res. (2008) 6:1928-1936); chronic myeloid leukemia (CML) (Dierks et al., Cancer Cell (2008) 14:238-249); colon cancer (Yang and Hinds, BMC Developmental Biology (2007) 7:6); esophageal cancer (Berman et al., Nature (2003) 425:846-851; WO 2005/013800); gastric cancer (Berman et al., Nature (2003) 425:846-851; Ma et al., Carcinogenesis (2005) 26:1698-1705; WO 2005/013800; Shiotani et al., J. Gastroenterol. Hepatol. (2008) S161-S166; Ohta et al., Cancer Research (2005) 65:10822-10829; Ma et al., World J. Gastroenterol (2006) 12:3965-3969); gastrointestinal stromal tumor (GIST) (Yoshizaki et al., World J. Gastroenterol (2006) 12:5687-5691); hepatocellular cancer (Sicklick et al., Carcinogenesis (2006) 27:748-757; Patil et al., Cancer Biology & Therapy (2006) 5:111-117); kidney cancer (Cutcliffe et al., Human Cancer Biology (2005) 11:7986-7994); lung cancer (Watkins et al., Nature (2003) 422:313-317); medulloblastoma (Berman et al., Science (2002) 297:1559-1561; Pietsch et al. Cancer Research (1997) 57:2085-2088); melanoma (Stecca et al., PNAS (2007) 104:5895-5900; Geng et al., Angiogenesis (2007) 10:259-267); multiple myeloma (Peacock et al., PNAS USA (2007) 104:4048-4053; Dierks et al., Nature Medicine (2007) 13:944-951); neuroectodermal tumors (Reifenberger et al., Cancer Research (1998) 58:1798-1803); non-Hodgkin's type lymphoma (NHL) (Dierks et al., Nature Medicine (2007) 13:944-951; Lindemann, Cancer Research (2008) 68:961-964); osteosarcoma (Warzecha et al., J. Chemother. (2007) 19:554-561); ovarian cancer (Steg et al., J. Molecular Diagnostics (2006) 8:76-83); pancreatic cancer (Thayer et al., Nature (2003) 425:851-856; Berman et al., Nature (2003) 425:846-851; WO 2005/013800); prostate cancer (Karhadkar et al., Nature (2004) 431:707-712; Sheng et al., Molecular Cancer (2004) 3:29-42; Fan et al., Endocrinology (2004) 145:3961-3970); and testicular cancer (Dormeyer et al., J. Proteome Res. (2008) 7:2936-2951).

### Combination Therapy

It will be appreciated that the pharmaceutical composition, as described above and herein, can be administered in combination with one or more additional therapies, e.g., such as radiation therapy, surgery and/or in combination with one or more therapeutic agents, to treat the cancers described herein.

By “in combination with,” it is not intended to imply that the therapy or the therapeutic agents must be administered at the same time and/or formulated for delivery together, although these methods of delivery are within the scope of the invention. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination may be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination will be lower than those utilized individually.

In certain embodiments, the cancer treated by the methods described herein can be selected from, for example, medulloblastoma, chondrosarcoma, osteosarcoma, pancreatic cancer, lung cancer (e.g., small cell lung cancer (SCLC) or non-small cell lung cancer (NSCLC)), ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, and prostate cancer.

An example of suitable therapeutics for use in combination with the pharmaceutical compositions of the invention for treatment of medulloblastoma includes, but is not limited to, a chemotherapeutic agent (e.g., lomustine, cisplatin, carboplatin, vincristine, and cyclophosphamide), radiation therapy, surgery, and a combination thereof.

An example of suitable therapeutics for use in combination with the pharmaceutical compositions of the invention for treatment of chondrosarcoma includes, but is not limited to, a chemotherapeutic agent (e.g., trabectedin), radiation therapy (e.g., proton therapy), surgery, and a combination thereof.

An example of suitable therapeutics for use in combination with the pharmaceutical compositions of the invention for treatment of osteosarcoma includes, but is not limited to, a chemotherapeutic agent (e.g., methotrexate (e.g., alone or in combination with leucovorin rescue), cisplatin, adriamycin, ifosfamide (e.g., alone or in combination with mesna), BCG (Bacillus Calmette-Guerin), etoposide, muramyl tri-peptite (MTP)), radiation therapy, surgery, and a combination thereof.

An example of suitable therapeutics for use in combination with the pharmaceutical compositions of the invention for treatment of pancreatic cancer includes, but is not limited to, a chemotherapeutic agent, e.g., paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); RaFMEK dual kinase inhibitor (e.g., RO5126766); Notch signaling inhibitor (e.g., MK0752); monoclonal antibody-antibody fusion protein (e.g., L19IL2); curcumin; HSP90 inhibitor (e.g., IPI-493, IPI-504, tanespimycin, STA-9090); rIL-2;, denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., RO4929097); ribonucleotide reductase inhibitor (e.g., 3-AP); immunotoxin (e.g., HuC242-DM4); PARP inhibitor (e.g., KU-0059436, veliparib); CTLA-4 inhbitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof In certain embodiments, a combination of paclitaxel or a paclitaxel agent, and gemcitabine can be used with the pharmaceutical compositions of the invention.

An example of suitable therapeutics for use in combination with the pharmaceutical compositions of the invention for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., IPI-493, IPI-504, tanespimycin, STA-9090); mTOR inhibitor (e.g., everolimus); Ep-CAM-/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof

An example of suitable therapeutics for use in combination with the pharmaceutical compositions of the invention for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, RO5083945), MET inhibitor (e.g., PF-02341066, ARQ 197), PI3K kinase inhibitor (e.g., XL147, GDC-0941), Raf/MEK dual kinase inhibitor (e.g., RO5126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine , GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., IPI-493, IPI-504, tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., RO4929097), epigenetic therapy (e.g., 5-azacitidine), nitroglycerin, MEK inhibitor (e.g., AZD6244), cyclin-dependent kinase inhibitor (e.g., UCN-01), cholesterol-Fusl, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination with the pharmaceutical compositions of the invention for treatment of ovarian cancer includes, but is not limited to, a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agnet (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., RO4929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy; and combinations thereof

An example of suitable therapeutics for use in combination with the pharmaceutical compositions of the invention for treatment of chronic myelogenous leukemia (AML) according to the invention includes, but is not limited to, a chemotherapeutic (e.g., cytarabine, hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib, RGB-286638)), interferon alfa, steroids, apoptotic agent (e.g., omacetaxine mepesuccinat), immunotherapy (e.g., allogeneic CD4+ memory Thl-like T cells/microparticle-bound anti-CD3/anti-CD28, autologous cytokine induced killer cells (CIK), AHN-12), CD52 targeting agent (e.g., alemtuzumab), HSP90 inhibitor (e.g., IPI-493, IPI-504, tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., RO5045337), Aurora B kinase inhibitor (e.g., TAK-901), radioimmunotherapy (e.g., actinium-225-labeled anti-CD33 antibody HuM195), Hedgehog inhibitor (e.g., PF-04449913), STAT3 inhibitor (e.g., OPB-31121), KB004, cancer vaccine (e.g., AG858), bone marrow transplantation, stem cell transplantation, radiation therapy, and combinations thereof.

An example of suitable therapeutics for use in combination with the pharmaceutical compositions of the invention for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., ABT-263), immunotherapy (e.g., allogeneic CD4+ memory Thl -like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., RO5045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., 1NCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination with the pharmaceutical compositions of the invention for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., RO5045337), immunotoxin (e.g., CAT-8015, DT2219ARL), HDAC inhibitor (e.g., JNJ-26481585), JVRS-100, paclitaxel or a paclitaxel agent, STATS inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination with the pharmaceutical compositions of the invention for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT388IL3 fusion protein, HDAC inhibitor (e.g., vorinostat, LBH589), plerixafor, mTOR inhibitor (e.g., everolimus), SRC inhibitor (e.g., dasatinib), HSP90 inhbitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., RO5045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of suitable therapeutics for use in combination with the pharmaceutical compositions of the invention for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3 inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of suitable therapeutics for use in combination with the pharmaceutical compositions of the invention for treatment of head and neck cancer includes, but is not limited to, a chemotherapeutic (e.g., paclitaxel or a paclitaxel agent, carboplatin, docetaxel, amifostine, cisplantin, oxaliplatin, docetaxel), tyrosine kinase inhibitors (e.g., EGFR inhibitor (e.g., erlotinib, gefitinib, icotinib, cetuximab, panitumumab, zalutumumab, nimotuzumab, necitumumab, matuzumab, cetuximab), dual inhibitor (e.g., lapatinib, neratinib, vandetanib, BIBW 2992, multikinase inhibitor (e.g., XL-647)), VEGF inhibitor (e.g., bevacizumab), reovirus, radiation therapy, surgery, and a combination thereof.

An example of suitable therapeutics for use in combination with the pharmaceutical compositions of the invention for treatment of prostate cancer includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

In some embodiments, the pharmaceutical composition described herein is used in combination with a mTOR inhibitor, e.g., one or more mTOR inhibitors chosen from one or more of rapamycin, temsirolimus (TORISEL®), everolimus (RAD001, AFINITOR®), ridaforolimus, AP23573, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, or PKI-587. In one embodiment, the mTOR inhibitor inhibits TORC1 and TORC2. Examples of TORC1 and TORC2 dual inhibitors include, e.g., OSI-027, XL765, Palomid 529, and INK128.

In some embodiments, the pharmaceutical composition described herein is used in combination with an inhibitor of insulin-like growth factor receptor (IGF-1R), e.g., BMS-536924, GSK1904529A, AMG 479, MK-0646, cixutumumab, OSI 906, figitumumab (CP-751,871), or BIIB022.

In some embodiments, the pharmaceutical composition described herein is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074,nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In some embodiments, the pharmaceutical composition described herein is used in combination with folfirinox comprising oxaliplatin 85 mg/m2 and irinotecan 180 mg/m2 plus leucovorin 400 mg/m2 followed by bolus fluorouracil (5-FU) 400 mg/m2 on day 1, then 5-FU 2,400 mg/m2 as a 46-hour continuous infusion.

In some embodiments, the pharmaceutical composition described herein is used in combination with a PI3K inhibitor. In one embodiment, the PI3K inhibitor is an inhibitor of delta and gamma isoforms of PI3K. Exemplary PI3K inhibitors that can be used in combination are described in, e.g., WO 2010/036380; WO 2010/006086, WO 09/114870, WO 05/113556. Additional PI3K inhibitors that can be used in combination with the pharmaceutical compositions, include but are not limited to, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof

In some embodiments, the pharmaceutical composition described herein is administered in combination with a BRAF inhibitor, e.g., GSK2118436, RG7204, PLX4032, GDC-0879, PLX4720, and sorafenib tosylate (Bay 43-9006).

In some embodiments, the pharmaceutical composition described herein is administered in combination with a MEK inhibitor, e.g., ARRY-142886, GSK1120212, RDEA436, RDEA119/BAY 869766, AS703026, AZD6244 (selumetinib), BIX 02188, BIX 02189, CI-1040 (PD184352), PD0325901, PD98059, and U0126.

In some embodiments, the pharmaceutical composition described herein is administered in combination with a JAK2 inhibitor, e.g., CEP-701, 1NCB18424, CP-690550 (tasocitinib)

In some embodiments, the pharmaceutical composition described herein is administered in combination with paclitaxel or a paclitaxel agent, e.g., TAXOL®, protein-bound paclitaxel (e.g., ABRAXANE®). A “paclitaxel agent” as used herein refers to a formulation of paclitaxel (e.g., for example, TAXOL) or a paclitaxel equivalent (e.g., for example, a prodrug of paclitaxel). Exemplary paclitaxel equivalents include, but are not limited to, nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., T-paclitaxel methyl 2-glucopyranosyl succinate, see Liu et al., Bioorganic & Medicinal Chemistry Letters (2007) 17:617-620). In certain embodiments, the paclitaxel agent is a paclitaxel equivalent. In certain embodiments, the paclitaxel equivalent is ABRAXANE. In some embodiments, the pharmaceutical composition described herein is administered in combination with a paclitaxel agent and gemcitabine.

### Exemplification

The following examples are included merely for purposes of illustration and are not intended to limit the disclosure herein.

Direct Blend Formulations

Early dissolution experiments on IPI-926 encapsulated in a gelatin capsule resulted in an insoluble solid agglomerate. Little to no exposure of IPI-926 was observed upon administration of this formulation to Beagle dogs. Without wishing to be bound to any particular theory, it was hypothesized that IPI-926 formed the solid agglomerate upon hydration. Accordingly, direct blend formulations were investigated.

A filler such as microcrystalline cellulose is well-suited as a direct blend with IPI-926 due to its surface and adhesion characteristics and its insolubility in water. The microcrystalline cellulose filler, Avicel™ PH-200, available from FMC Corporation, has a rough porous surface, a non-spherical shape, and a nominal particle size of about 180 microns with a low proportion of fines, less than 25% by weight of particles smaller than 125 microns.

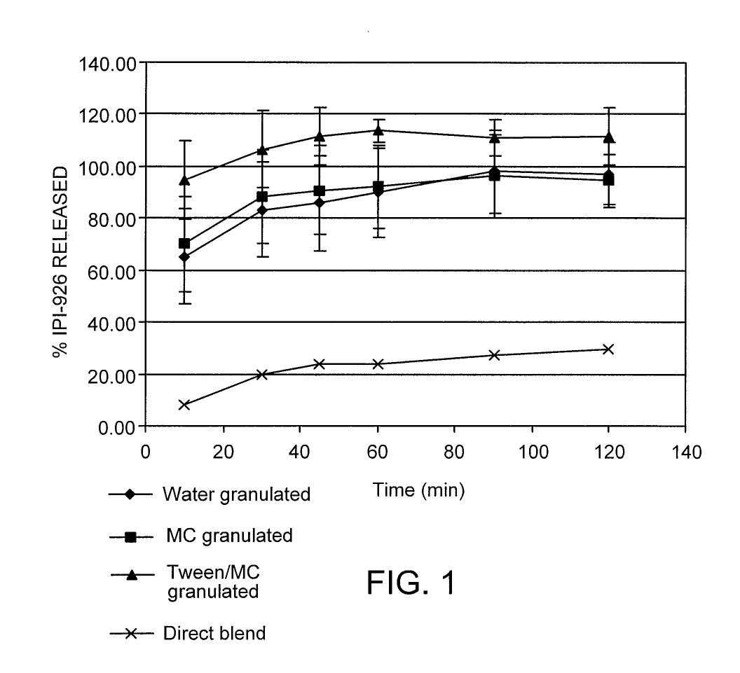

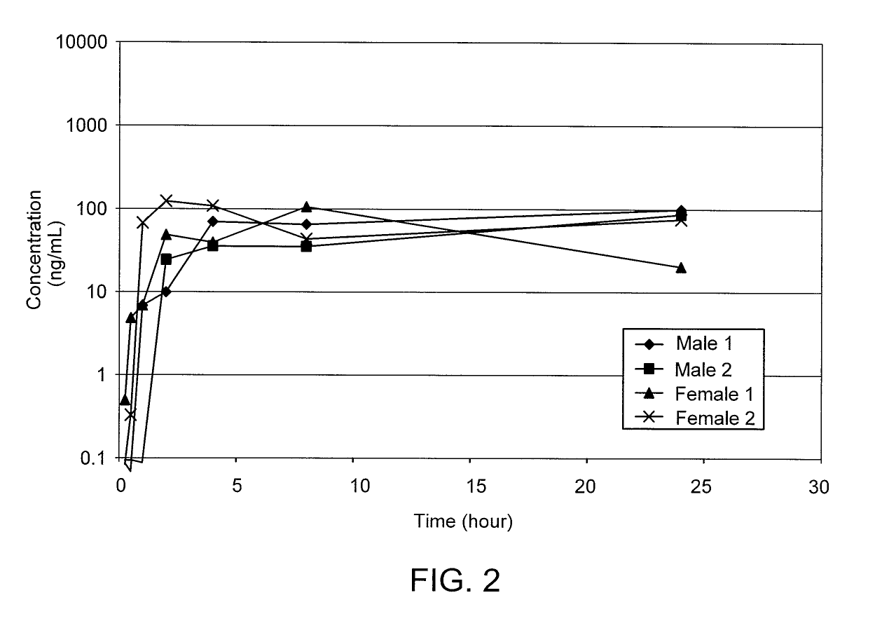

In one experiment, 40% w/w IPI-926 and 60% w/w Avicel™ PH-200 were blended in a Turbula blender and then manually filled into gelatin capsules. This formulation showed an improved dissolution profile compared to other IPI-926 formulations (as described above), but still displayed less than 30% release after 1 hour (FIG. 1). FIG. 2 depicts the exposure of IPI-926 in male and female Beagle dogs after administration this formulation (4 mg/kg/day).

II. Suspension Formulations

To facilitate dosing by oral gavage, suspension formulations of IPI-926 were prepared. These suspensions were typically prepared by wetting IPI-926 with a levigating agent such as polysorbate 80 (Tween-80) and agitating the wet mixture in the presence of an aqueous suspending agent such as methylcellulose. The resultant suspensions formed either a viscous foam or a solid agglomerate. Use of different levigating agents, such as glycerol, or different suspending agents such as aqueous sodium carboxymethylcellulose or a mixture thereof, continued to provide formulations which formed a foam or solid agglomerate. Upon homogenization, these suspensions were found to have excessive aggregates of large crystals and further gelling. Additional mixing, vortexing, and/or sonication did not resolve this issue.

It was later found that addition of a Tween surfactant (e.g., Tween-80) to the suspending agent prior to levigation reduced foaming, settling, agglomeration and/or precipitation, and provided a free-flowing, well-distributed suspension.

In one experiment, it was found that between about 5% and about 10% Tween-80 added to a 1% methylcellulose suspension of IPI-926 reduced the foaming, and provided a uniform viscous suspension. A series of formulations were made to minimize the level of excipients while still maintaining the desired suspension properties.

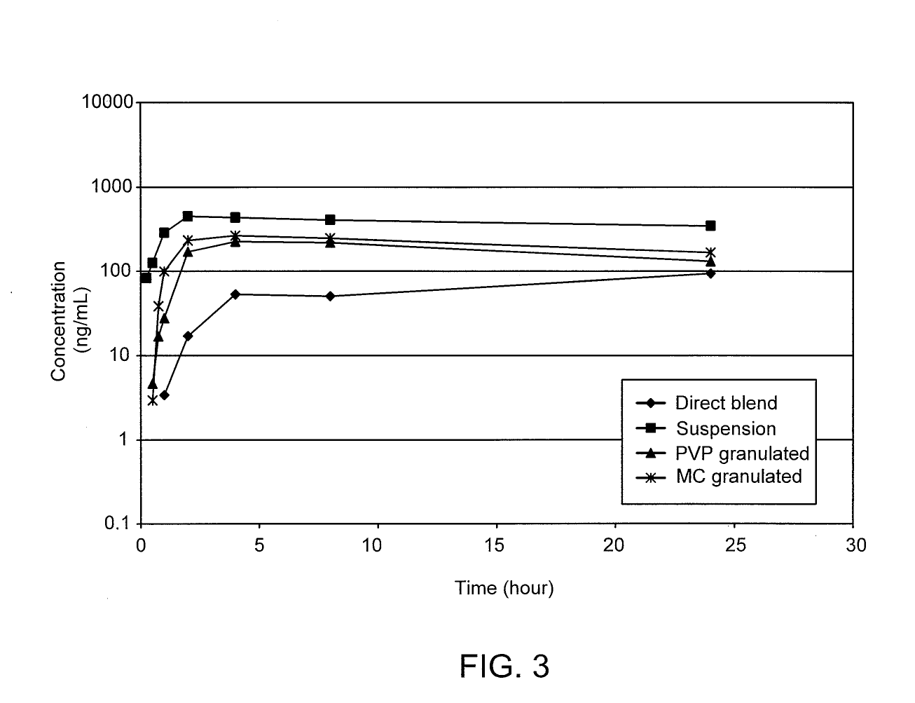

A suspension of IPI-926 in 0.25% methylcellulose, 2.5% Tween-80, and 97.25% water administered to male Beagle dogs (4 mg/kg) displayed good exposure (see FIG. 3).

III. Granulated Formulations

Granulated formulations were also explored as granulation processes typically increase blend uniformity and flowability and aid in drug product processing (e.g., capsule filling).

In one study, methylcellulose granulation formulations of IPI-926 were explored.

IPI-926 was weighed and screened through a #20 sieve. Water or an aqueous solution of methylcellulose or a 10:1 mixture of Tween-80: methylcellulose was added by transfer pipette to a visual end point wherein IPI-926 appeared granulated. The wet granulation was then dried overnight in a convection oven at 50° C. After drying, the granulation was passed through a #20 mesh. 40% w/w of the granulation was then blended with 60% w/w of Avicel™ PH-200 for 5 minutes on a Turbula Blender. The final blend was manually filled into gelatin capsules to 30 mg of IPI-926.

This procedure provided the following granulated formulations: a granulated IPI-926 formulation from water+blend of Avicel™ PH-200 (water granulated), a 97% w/w IPI-926+3% w/w methylcellulose granulated formulation blend of Avicel™ PH-200 (MC granulated), and a 96.7% w/w IPI-926+3.0% Tween-80+0.3% w/w methylcellulose granulated formulation blend of Avicel™ PH-200 (Tween/MC granulated). Each of these formulations provided from this procedure each displayed similar release profiles (FIG. 1). In general, it was found that the dissolution profile improved with granulation compared to the 40% w/w IPI-926 and 60% w/w Avicel™ PH-200 direct blend formulation. Both the methylcellulose and the Tween/methylcellulose granulated formulations provided better granule strength than the water granulated formulation, most likely due to the inclusion of a binder.

In another study, PVP granulation formulations of IPI-926 were explored. For the PVP granulation, 97% w/w of IPI-926 and 3% w/w PVP-K30 were weighed and screened through a #20 sieve. Water was then added with a transfer pipette to a visual end point when the blend appeared granulated. The granulation was then dried overnight in a convection oven at 50° C. After drying, the granulation was passed through a #20 mesh. 40% w/w of the granulation was then blended with 60% w/w of Avicel™ PH-200 for 5 minutes on a Turbula Blender. The final blend was manually filled into gelatin capsules to 30 mg of IPI-926.

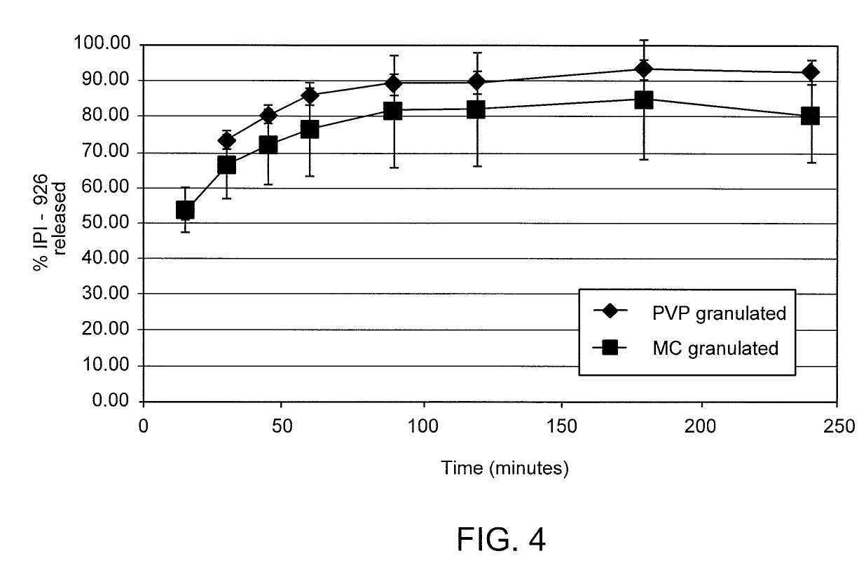

Dissolution studies indicated that the granulated PVP formulation provided from this procedure displayed a similar release profile to the granulated methylcellulose formulation as described above (FIG. 4). The PVP and the methylcellulose granulation formulations and the IPI-926 in Avicel™ PH-200 direct blend formulation were dosed in male Beagle dogs (4 mg/kg), and demonstrated similar exposure (FIG. 3).

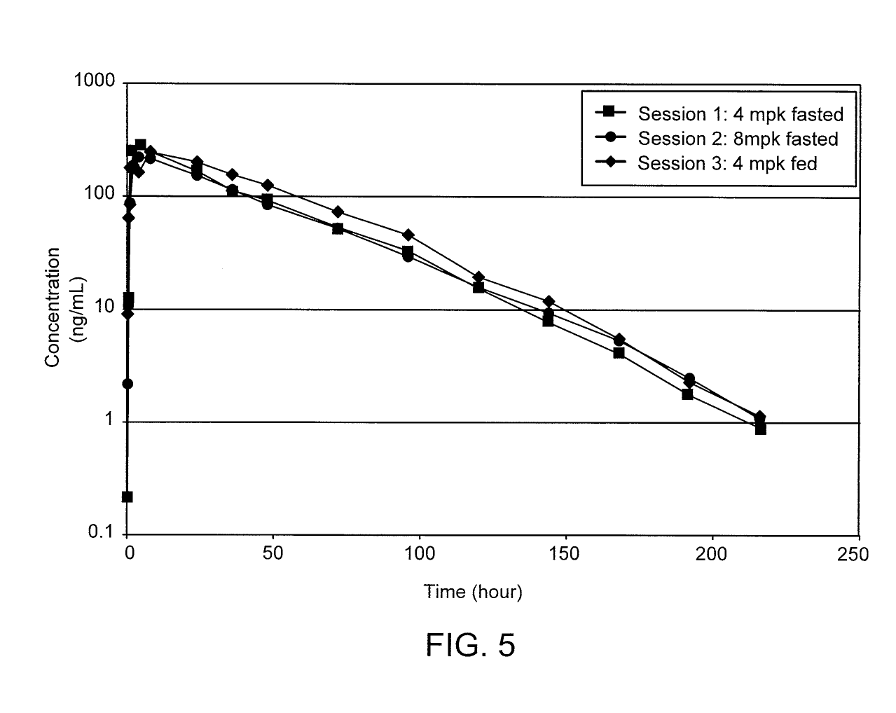

Additional in vivo studies were conducted with PVP granulation formulations due to its ease of processing. The PVP granulation formulation was dosed into beagle dogs at 4 mg/kg fasted and 8 mg/kg fasted to see if the formulation displayed dose-proportional exposure. It was also dosed at 4 mg/kg fed in order to establish a fed/fasted correlation. All sessions had at least 10 day washout in between sessions. Sessions used the same dogs with an average weight of 7 kg. The capsules were filled individually to match dog weight. The results of this study are summarized in Table 1 and FIG. 5.

TABLE 1

| | | | IPI- | | |
|---|---|---|---|---|---|
| | | | 926 | | |
| | Study | | (active | AUC₂₄ | C_{MAX} |
| Session | day | Route* | moiety) | (mean) | (mean) |
| 1 | 1 | Oral | 4 mg/kg/day | 11957 | 285 |
| | | (Gelcap) | | | |
| 2 | 15 | Oral | 8 mg/kg/day | 11022 | 241 |
| | | (Gelcap) | | | |
| 3 | 29 | Oral | 4 mg/kg/day | 14204 | 306 |
| | | (Gelcap) | FED | | |

*administration to 2 male and 2 female Beagle dogs

Exposures were similar for the beagles dosed at 4 mg/kg fed and 4 mg/kg fasted. However, dogs dosed at 4 mg/kg and 8 mg/kg did not display dose-proportional exposure (see FIG. 5).

In another study, the PVP formulation was modified in order to increase solubility of the formulation and disintegration properties of the capsule. Tween 80 was added to the granulating solution to increase solubility and permeability in-vivo. Avicel™ PH-200 was added to the granulation step in order to aid in the granulation process and facilitate the drying of the granulated mixture. Less extragranular Avicel™ PH-200 was required to reduce particle segregation, and less IPI-926 was added in order to get better content uniformity, and to increase the release rate by decreasing disintegration time.

In an exemplary procedure, an aqueous Tween-80 solution was added to a planetary Mixer containing IPI-926, PVP-K30 and Avicel™ PH-200 until a visual end point indicated the blend granulated. The granulation was then tray dried in a forced convection oven at 40° C. to <5% water and manually passed through a #20 mesh or milled in a Comil. An HPLC assay was performed on dried granulation to determine the capsule fill weight based on resulting potency. Extragranular Avicel™ PH-200 was optionally blended with the granulation. The dried granulation was then filled into HPMC capsules using a Minicap 100 capsule filler and the capsules were stored at 5° C. This process was used to refine the formulation and generate development batches prior to manufacturing clinical trial materials.

Pilot batches of capsules were produced at 3 strengths: 10 mg, 30 mg and 120 mg (Table 2). A low potency granulation was manufactured for 10 and 30 mg capsules, and a high potency granulation was manufactured for 120 mg capsules.

TABLE 2

| | 10 mg (low | | 30 mg (low | | 120 mg (high | |
|---|---|---|---|---|---|---|
| | potency) | | potency) | | potency) | |
| | % w/w | mg/cap | % w/w | mg/cap | % w/w | mg/cap |
| Active IPI- | 9.30 | 10.0 | 8.70 | 30.0 | 26.09 | 120.0 |
| 926* | | | | | | |
| Avicel ™ | 81.72 | 87.9 | 76.42 | 263.6 | 58.23 | 267.8 |
| PH-200* | | | | | | |
| (intragranular) | | | | | | |
| PVP K- | 2.81 | 3.0 | 2.63 | 9.1 | 2.65 | 12.2 |
| 30 | | | | | | |
| Tween | 6.16 | 6.6 | 5.76 | 19.9 | 5.02 | 23.1 |
| 80 | | | | | | |
| Avicel ™ | — | — | 6.49 | 22.4 | 8.01 | 36.8 |
| PH-200 | | | | | | |
| (extragranular) | | | | | | |
| Total | 100.00 | 107.5 | 100.00 | 345.0 | 100.01 | 459.9 |

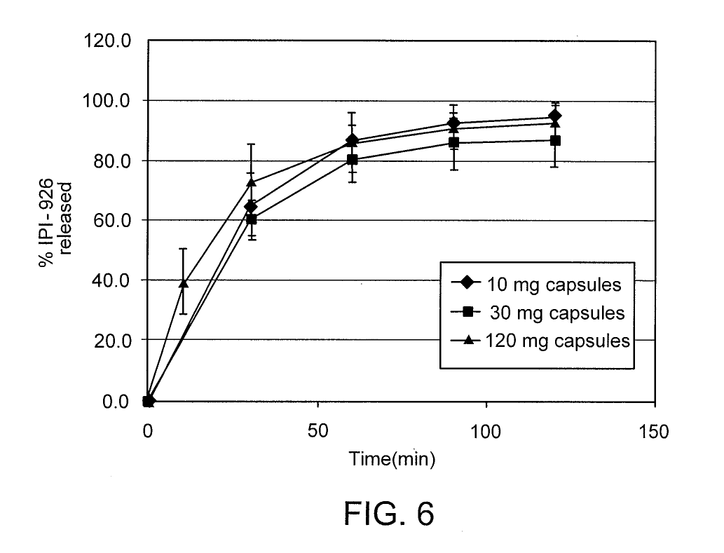

Dissolution testing of 10 mg and 30 mg capsules exhibited good release profiles (FIG. 6). Surprisingly, the 120 mg capsules did not meet the dissolution specification of Q=75% in 90 minutes. It was hypothesized that in these capsules localized agglomeration occurred and the reduced surface area of the agglomeration hindered dissolution. In order to overcome the lack of disintegration, formulations were explored using croscarmellose sodium (AcDiSol™) as a disintegrant. AcDiSol™ was added both intagranularly and extragranularly in the final clinical formulation of the 120 mg capsule (Table 3).

TABLE 3

Clinical 120 mg
formulation (high potency)

| | % w/w | wt. per capsule (mg) |
|---|---|---|
| Active IPI-926* | 26.09 | 120.0 |
| Avicel ™ PH-200* | 52.80 | 243.0 |
| (intragranular) | | |
| AcDiSol ™ (intragranular) | 3.61 | 16.6 |
| PVP K-30 | 2.59 | 11.9 |
| Tween 80 | 6.89 | 31.7 |
| AcDiSol ™ (extragranular) | 8.00 | 36.8 |
| Total | 100.00 | 460.0 |

Twelve capsules were tested for the 10 and 30 mg capsule strengths and six capsules were tested for the 120 mg capsule strength. 10 and 30 mg showed acceptable release profiles. 120 mg capsules containing formulation of Table 3 exhibited less capsule to capsule variability as well as more complete release than the formulation of Table 2 due to improved disintegration of the capsule contents. Both formulations displayed acceptable dissolution profiles. All three capsule strengths exhibit a similar release profile (FIG. 6).

Additional formulations of IPI-926 with acceptable dissolution profiles are shown in Tables 4, and 5.

TABLE 4

| | |
|---|---|
| 10 mg | 30 mg |

| | % w/w | mg/cap | % w/w | mg/cap |
|---|---|---|---|---|
| Active IPI-926 | 9.3 | 10.0 | 8.7 | 30 |
| Avicel ™ PH-200 | 81.8 | 87.9 | 76.4 | 264 |
| (intragranular) | | | | |
| PVP K-30 | 2.8 | 3.0 | 2.6 | 9.0 |
| Tween 80 | 6.1 | 6.6 | 5.7 | 20 |
| Avicel ™ PH-200 | — | — | 6.5 | 22 |
| (extragranular) | | | | |

TABLE 5

| | % w/w | mg/cap |
|---|---|---|
| Active IPI-926 | 26.1 | 120.0 |
| Avicel ™ PH-200 | 52.8 | 243.0 |
| AcDiSol | 3.6 | 16.6 |
| (intragranular) | | |
| PVP K-30 | 2.6 | 11.9 |
| Tween 80 | 6.9 | 31.7 |
| AcDiSol (extragranular) | 8.0 | 36.8 |
| Total | 100 | 460.0 |

Particle Distribution Study. The formulations described in Table 4 were analyzed for particle size using the method described herein. The results are summarized in Table 6 below.

TABLE 6

| Screen # | Size (μm) | % w/w |
|---|---|---|
| 35 | 850-500 | 47.8 |
| 60 | 500-250 | 32.2 |
| 100 | 250-150 | 10.4 |
| Pan | <150 | 9.6 |

As can be seen, a significant amount of the formulation has a particle size >500 micrometers.

Dissolution by Particle Size. The formulation particle sizes obtained as described above (see Table 6) were analyzed for extent of dissolution using the method described herein. The results are summarized in Table 7 below, in which extend of dissolution is expressed as % IPI-926 release of

TABLE 7

| Time | 850-500 | 500-250 | | | |
|---|---|---|---|---|---|
| (minutes) | (μm) | (μm) | 250-150 (μm) | <150 (μm) | Composite |
| 15 | 10.0 | 20.6 | 45.3 | 48.2 | 15.3 |
| 30 | 54.6 | 61.5 | 59.1 | 46.9 | 69.5 |
| 45 | 79.6 | 76.7 | 61.7 | 42.0 | 85.2 |
| 60 | 87.1 | 75.1 | 72.2 | 52.0 | 89.6 |
| 90 | 101.1 | 75.5 | 69.7 | 55.1 | 96.0 |
| 120 | 111.1 | 84.0 | 63.9 | 49.0 | 100.5 |

As can be seen, the smallest sized particles exhibited a generally flat line release of about 50%. The largest sized particles exhibited a slower release, but significantly reduced potential for gelling.

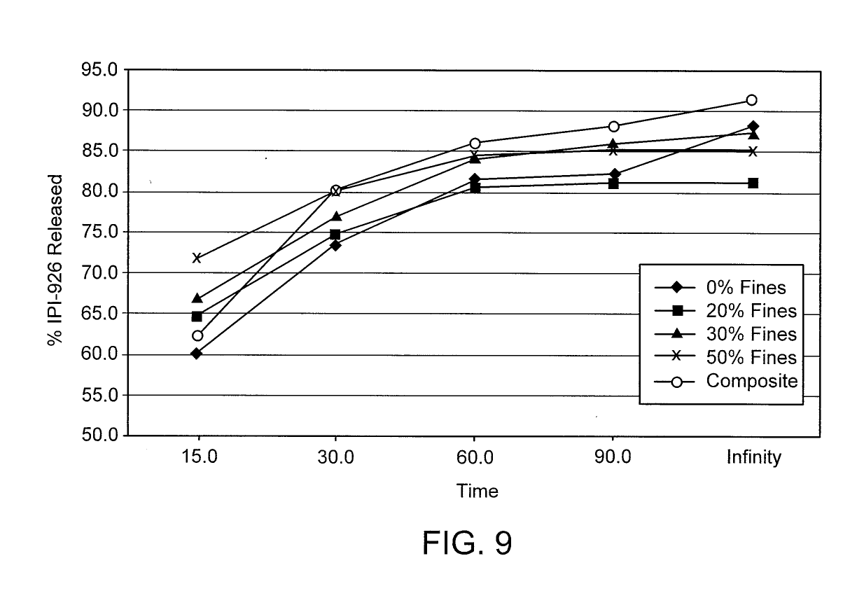

Tolerance of fines. The low potency clinical formulations described in Table 4 (a significant amount of these formulation were found to have a particle size >500 micrometers) were analyzed for tolerance of increasing the amount of fines using the dissolution protocol described herein. The formulation was reblended to include 20%, 30%, and 50% (w/w) of formulation having a particle size of <150 micrometers (i.e., a particle size which was shown to exhibit a generally flat line release of about 50%). The results, which are summarized in FIG. 9, indicate that the indicated levels of added fines was tolerated on the basis of dissolution performance.

Stability Studies. Two lots of the clinical formulations described in Table 3 were prepared by wet granulation using 57.3 and 64.5 weight percent water. Both lots were studied for storage stability using the methods described herein. Both lots exhibited comparable stability upon storage at -20° C. (3 days) and 5° C. (6 months). The lot obtained using 64.5 weight percent water was found to exhibit enhanced stability relative to the lot prepared with less water at both 25° C./60% relative humidity (6 months instead of 3 months) and 40° C./75% relative humidity (1 month instead of 3 days).

Determination of Crystallinity. Using the method described herein (residual IPA method), the crystallinity of the two lots described in the stability studies were determined. The crystallinity of the lots prepared using 57.3 and 64.5 weight percent water were found to be 6.0% and 1.4%, respectively. The GC conditions: Agilent J&W column, DB-624, 30 m×0.32 mm ID, 1.8 μmfilem thickness; helium carrier gas @ 0.9 mL/minute; 21 minute run time; injector temperature=150° C. with split ratio of 10:1; detection FID 300° C.; 30 mL/minute hydrogen; air 300 mL/minute; make up with helium 10 mL/minute; the initial column temperature is 40° C. increasing at 20° C. per minute to 120° C., hold for 5 minutes then increase 30° C. per minute to 240° C.; oven temperature 120 C; loop and transfer line temperature of 135° C. and 145° C., respectively.

Effect of Particle Size and Crystallinity on Dissolution. Table 8 shows the results of a dissolution study of 30 mg IPI-926 Capsules containing low (<1%) crystalline granulation. As can be seen, at least 75% release is achieved after 90 minutes for each of the different particle size distributions tested. Table 9 shows the results of a dissolution study of 30 mg IPI-926 Capsules containing high (<95%) crystalline granulation. As can be seen, 75% release is achieved after 90 minutes only in the case of the larger particle size (>500 micrometer) sample. Table 10 shows the results of a dissolution study of 10 mg IPI-926 Capsules containing low (<1%) crystalline granulation. As can be seen, at least 75% release is achieved after 90 minutes for the larger particle size (>500 micrometer) sample and the composite sample, but not the smaller particle size sample (<150 micrometer). Table 11 shows the results of a dissolution study of 10 mg IPI-926 Capsules containing high (<95%) crystalline granulation. As can be seen, 75% release is achieved after 90 minutes only in the case of the larger particle size (>500 micrometer) sample.

TABLE 8

| 30 mg IPI-926 Capsules, | % IPI-926 Released at 90 |
|---|---|
| <1% crystalline Granulation | minutes |
| <500 um Wet | 79 |
| Granulation | |
| >500 um Wet | 94 |
| Granulation | |
| Composite Wet | 92 |
| Granulation | |

TABLE 9

| | % IPI-926 |
|---|---|
| 30 mg IPI-926 Capsules, >95% | Released at 90 |
| Crystalline Dry Granulation | minutes |
| <500 Roller Compacted | 29 |
| >500 Roller Compacted | 80 |
| Roller Compacted Composite | 61 |

N = 4

TABLE 10

| 10 mg IPI- | % |
|---|---|
| 926 Capsules, <1% | IPI-926 |
| crystalline Wet | Released at |
| Granulation, n = 4 | 90 Minutes |
| >500 um | 100 |
| <150 um | 55 |
| Composite | 96 |

TABLE 11

| 10 mg IPI-926 Capsules, | % IPI- |
|---|---|
| >95% Crystalline Dry Granulation, | 926 Released at |
| n = 4 | 90 Minutes |
| >500 um Dry Granulation | 77 |
| <500 um Dry Granulation | 35 |

This example shows that low crystalline material tends to shows better dissolution profile than the corresponding high crystalline material. This example further shows that larger sized particles tend to exhibit better dissolution profiles than smaller sized particles. Finally, this example shows that the dissolution profile of high crystalline material can be enhanced when larger particle sizes are employed.

Tablet Formulations. Tablet formulations having acceptable dissolution profiles were also prepared (e.g., 200 mg, 250 mg, and 400 mg tablet). A representative example is shown below.

| 250 mg Tablet Granulation | | | |
|---|---|---|---|
| | | Wt. per Tablet | Theoretical |
| Component | % w/w | (mg) | Wt. (g) |
| Active IPI- | 33.23 | 304.4 | 3.653 |
| 926* | | | |
| Avicel pH | 55.16 | 505.2 | 6.063 |
| 200* | | | |
| AcDiSoL | 1.76 | 16.2 | 0.194 |
| (intra) | | | |
| PVP K-30 | 2.71 | 24.8 | 0.298 |
| Tween 80 | 7.13 | 65.3 | 0.784 |
| Gran Total | 100.00 | 915.8 | 10.990 |

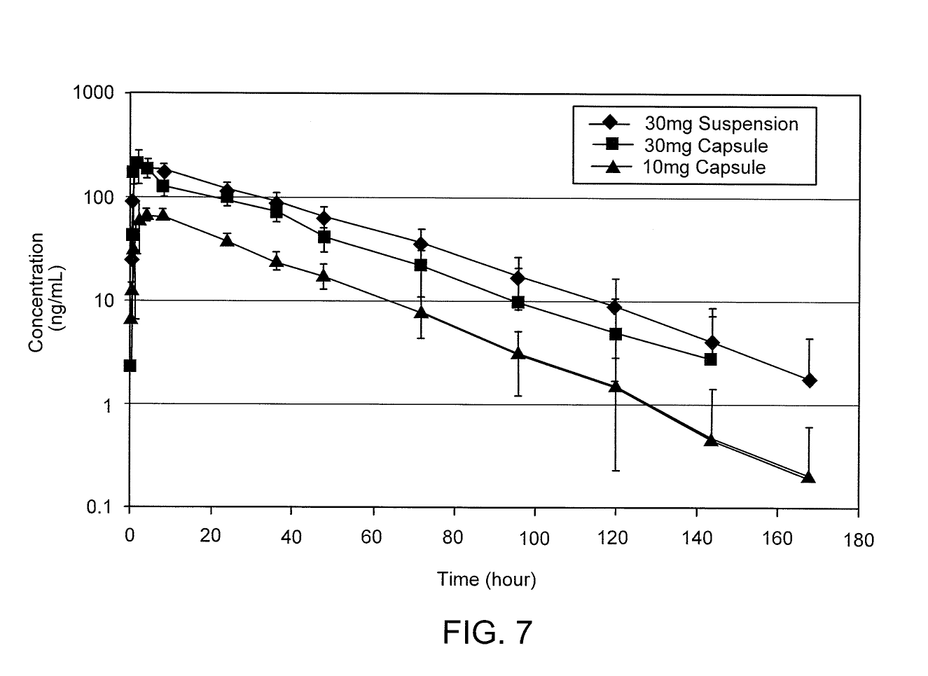

PK studies for 10 and 30 mg capsule strengths were performed on four fasted male and female Beagle dogs. Each dog was given a single 30 mg capsule. After an 8 day washout period, each dog was given a single 10 mg capsule. After a 12 day washout period, each dog was given 15 mL of a 2 mg/mL suspension of IPI-926 in 0.25% methylcellulose, 2.5% Tween-80, and 97.25% water by oral gavage. Blood samples were taken predose, 15 and 30 minutes, 1, 2, 4, 8, 24, 36, 48, 72, 96, 120, 144 and 168 hours post dose. The results of these experiments are summarized in FIG. 7 and Table 12.

TABLE 12

| | | | IPI-926 | | | | |
|---|---|---|---|---|---|---|---|
| | Study | | (active | | AUC₂₄ | C_{MAX} | T_{MAX} |
| Session | day | Route* | moiety) | | (mean) | (mean) | (mean) |
| 4 | 99 | Oral | 30 | mg/day | 8988 | 205 | 3.5 |
| | | (HPMC | | | | | |
| | | capsule) | | | | | |
| 5 | 108 | Oral | 10 | mg/day | 2478 | 72 | 4.0 |
| | | (HPMC | | | | | |
| | | capsule) | | | | | |
| 6 | 121 | Oral | 30 | mg/day | 8525 | 213 | 2.3 |
| | | (suspension) | | | | | |

*administration to 2 male and 2 female Beagle dogs

In order to compare the low potency and high potency formulations in Beagle dogs, a daily dose of 60 mg was selected to match the highest dose in a prior 4 week non-clinical toxicology study in mice.

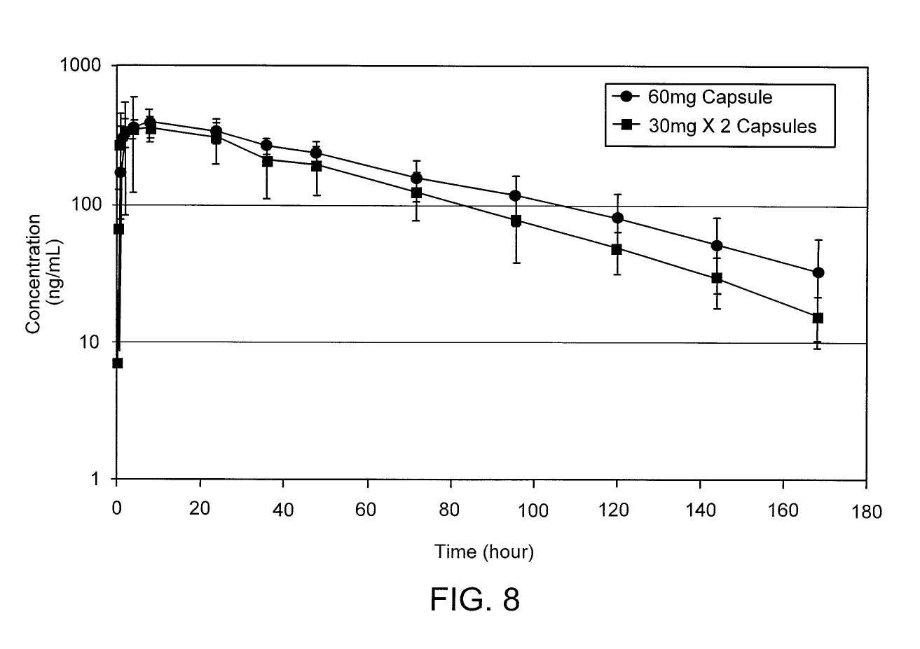

Four male Beagle dogs, fasted overnight, were given a single 60 mg dose containing half the dose of the clinical 120 mg granulation formulation (high potency). Following a two week washout period the same four dogs were dosed with two 30 mg capsules of the clinical 30 mg granulation formulation (low potency) after overnight fasting. Blood was collected for plasma analysis pre-dose and then 15, 30 minutes, 1, 2, 4, 8, 24, 36, 48, 72, 96, 120, 144, and 168 hours post dose. Each formulation demonstrated similar exposure in vivo (see Table 13 and FIG. 8).

TABLE 13

| | Study | | IPI-926 | AUC₂₄ | C_{MAX} | T_{MAX} |
|---|---|---|---|---|---|---|
| Session | day | Route* | (active moiety) | (mean) | (mean) | (mean) |
| 7 | 1 | Oral | 60 mg/day | 29859 | 455 | 9 |
| | | (HPMC | | | | |
| | | capsule) | | | | |
| 8 | 15 | Oral | 30 mg (× 2)/day | 23001 | 380 | 4 |
| | | (HPMC | | | | |
| | | capsule) | | | | |

*administration to 4 male Beagle dogs

### Equivalents

Those skilled in the art will recognize, or be able to ascertain using no more than routine experimentation, many equivalents to the specific embodiments of the invention described herein. Such equivalents are intended to be encompassed by the following claims.

1. A pharmaceutical formulation comprising a compound of formula (I): 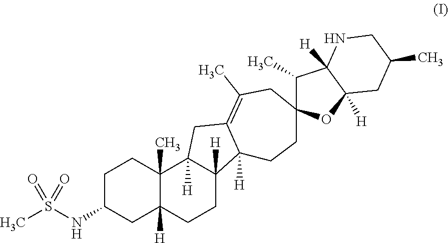
or a pharmaceutically acceptable salt thereof, wherein not more than 80% of the formulation have a particle size of less than 250 micrometers and wherein the formulation is in a form that is suitable for oral administration.

2. The formulation of claim 1, wherein from 10 percent to 60 percent of the formulation have a particle size of less than 250 micrometers.

3. The formulation of claim 1, wherein from 10 percent to 30 percent of the formulation have a particle size of less than 250 micrometers.

4. The formulation of claim 1, wherein from 20 percent to 90 percent of the formulation have a particle size that is greater than or equal to 250 micrometers.

5. The formulation of claim 4, wherein from 30 percent to 80 percent of the formulation have a particle size that is greater than or equal to 500 micrometers.

6. The formulation of claim 1, wherein from 40 percent to 90 percent of the formulation have a particle size that is greater than or equal to 250 micrometers.

7. The formulation of claim 6, wherein from 40 percent to 80 percent of the formulation have a particle size that is greater than or equal to 500 micrometers.

8. The formulation of claim 1, wherein from 10 percent to 60 percent of the formulation have a particle size of less than 250 micrometers; and from 40 percent to 90 percent of the formulation have a particle size that is greater than or equal to 250 micrometers.

9. The formulation of claim 8, wherein from 40 percent to 80 percent of the formulation have a particle size that is greater than or equal to 500 micrometers.

10. The formulation of claim 1, wherein the formulation has a particle size of at most about 1000 micrometers.

11. The formulation of claim 10, wherein from 20 percent to 90 percent of the formulation have a particle size of from 250 micrometers to 1000 micrometers.

12. The formulation of claim 11, wherein from 30 percent to 70 percent of the formulation have a particle size of from 500 micrometers to 1000 micrometers.

13. The formulation of claim 10, wherein from 40 percent to 90 percent of the formulation have a particle size of from 250 micrometers to 1000 micrometers.

14. The formulation of claim 13, wherein from 40 percent to 80 percent of the formulation have a particle size of from 500 micrometers to 1000 micrometers.

15. The formulation of claim 14, wherein from 40 percent to 80 percent of the formulation have a particle size of from 500 micrometers to 850 micrometers.

16. The formulation of claim 10, wherein from 10 percent to 60 percent of the formulation have a particle size of less than 250 micrometers; and from 40 percent to 90 percent of the formulation have a particle size of from 250 micrometers to 1000 micrometers.

17. The formulation of claim 16, wherein from 40 percent to 80 percent of the formulation have a particle size of from 500 micrometers to 1000 micrometers.

18. The formulation of claim 17, wherein from 40 percent to 80 percent of the formulation have a particle size of from 500 micrometers to 850 micrometers.

19. An oral pharmaceutical dosage formulation, comprising a compound of formula (I): 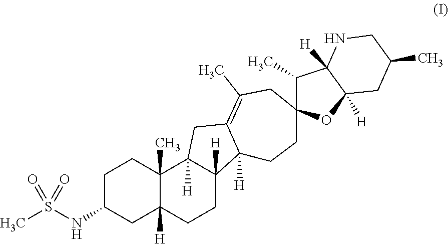
or a pharmaceutically acceptable salt thereof, wherein said compound is greater than 80% crystalline and at least 50% of particles of said formulation have a particle size of greater than 500 micrometers and wherein the formulation is in a form that is suitable for oral administration.

20. The formulation of claim 19, wherein at least 60% of particles of said formulation have a particle size of greater than 500 micrometers.

21. The formulation of claim 19, wherein at least 80% of particles of said formulation have a particle size of greater than 500 micrometers.

22. An oral pharmaceutical dosage formulation, comprising a compound of formula (I): 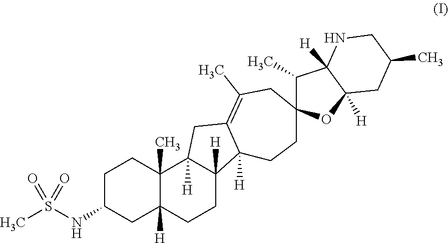
or a pharmaceutically acceptable salt thereof, wherein said compound is less than 80% crystalline and at least 20% of particles of said formulation have a particle size of greater than 250 micrometers and wherein the formulation is in a form that is suitable for oral administration.

23. The formulation of claim 22, wherein at least 40% of particles of said formulation have a particle size of greater than 250 micrometers.

24. The formulation of claim 22, wherein at least 50% of particles of said formulation have a particle size of greater than 250 micrometers.

25. The formulation of claim 22, wherein at least 20% of particles of said formulation have a particle size of greater than 500 micrometers.

26. The formulation of claim 22, wherein at least 50% of particles of said formulation have a particle size of greater than 500 micrometers.

27. The formulation of claim 1, wherein when the formulation is stirred at 37° C. in a dissolution media selected from 0.1 N aqueous HCl and 0.1 N aqueous HCl/0.5% Tween and at an maximum concentration selected from 0.011 mg of the compound of formula (I)/mL of dissolution media, 0.033 mg of the compound of formula (I)/mL of dissolution media, and 0.133 mg of the compound of formula (1)/mL of dissolution media, dissolution of the compound of formula (I) is at least 75% complete after 90 minutes as determined by HPLC.

28. The formulation of claim 1, wherein when the formulation is stable upon actual or simulated storage at 5° C. for at least 6 months.

29. The formulation of claim 1, wherein when the formulation is stable upon actual or simulated storage at 25° C./60% relative humidity for at least 3 months.

30. The formulation of claim 1, wherein when the formulation is stable upon actual or simulated storage at 40° C./75% relative humidity for 1 month.

31. The formulation of claim 1, wherein administration of a single dose of the formulation to a beagle dog produces a mean peak plasma concentration (Cmax) of the compound of formula (I) of between about 190 and 220 ng/mL for a formulation containing 30 mg of the compound of formula (I) and between about 60 and 80 ng/mL for a formulation containing 10 mg of the compound of formula (I).

32. The formulation of claim 1, wherein daily administration of the formulation to a human produces a mean steady state area under the concentration time curve (AUC_{(0-24 hrs)}) of the compound of formula (I) of between 5000 and 15,000 nghr/mL.

33. The formulation of claim 1, wherein the formulation comprises between 5% and 50% (w/w) of the active compound of formula (I).

34. The formulation of claim 33, wherein the formulation comprises between 5% and 15% (w/w) of the active compound of formula (I).

35. The formulation of claim 1, wherein the formulation comprises from 5 milligrams to 500 milligrams of the active compound of formula (I).

36. The formulation of claim 35, wherein the formulation comprises 10 milligrams or 30 milligrams of the active compound of formula (I).

37. The formulation of claim 33, wherein the formulation comprises between 20% and 30% (w/w) of the active compound of formula (I).

38. The formulation of claim 1, wherein the formulation comprises from 110 milligrams to 130 milligrams of the active compound of formula (I).

39. The formulation of claim 38, wherein the formulation comprises 120 milligrams of the active compound of formula (I).

40. The formulation of claim 1, wherein the compound of formula (I) is the hydrochloride salt.

41. The formulation of claim 1, wherein the formulation is a solid dosage form.

42. The formulation of claim 41, wherein the solid dosage form is a capsule or tablet.

43. The formulation of claim 42, wherein the solid dosage form is a capsule.

44. The formulation of claim 43, wherein the capsule is a gelatin capsule or a hydroxypropyl methylcellulose capsule.

45. The formulation of claim 1, wherein the formulation further comprises a filler.

46. The formulation of claim 45, wherein the filler is selected from microcrystalline cellulose, lactose, compressible sugar, pregelatinized starch, dibasic calcium phosphate, tribasic calcium phosphate, and calcium sulfate.

47. The formulation of claim 45, wherein the filler is microcrystalline cellulose.

48. The formulation of claim 1, wherein the formulation further comprises a binder.

49. The formulation of claim 48, wherein the binder is selected from polyvinylpyrrolidone, hydroxypropyl cellulose, methylcellulose, hydroxypropyl methylcellulose, pregelatizined starch, sucrose, and acacia gum.

50. The formulation of claim 49, wherein the binder is polyvinylpyrrolidone.

51. The formulation of claim 1, wherein the formulation further comprises a surfactant.

52. The formulation of claim 51, wherein the surfactant is selected from Tween 80, Tween 20, sodium laurel sulfate and sodium dodecyl sulfate.

53. The formulation of claim 52, wherein the surfactant is Tween 80.

54. The formulation of claim 1, wherein the formulation further comprises a disintegrant.

55. The formulation of claim 54, wherein the disintegrant is selected from croscarmellose sodium, sodium starch glycolate, crospovidone, and starch.

56. The formulation of claim 1, wherein the formulation further comprises microcrystalline cellulose, polyvinylpyrrolidine and Tween 80.

57. The formulation of claim 56, wherein the formulation further comprises croscarmellose sodium.

58. The formulation of claim 1, wherein the formulation comprises:

| | % w/w | mg/cap |
|---|---|---|
| Active Compound of | 9.3 | 10.0 |
| formula (I) | | |
| Avicel ™ PH-200 | 81.8 | 87.9 |
| (intragranular) | | |
| PVP K-30 | 2.8 | 3.0 |
| Tween 80 | 6.1 | 6.6 |
| Total | 100 | 107.5 |

59. The formulation of claim 1, wherein the formulation comprises:

| | % w/w | mg/cap |
|---|---|---|
| Active Compound of | 8.7 | 30.0 |
| formula (I) | | |
| Avicel ™ PH-200 | 76.4 | 263.7 |
| (intragranular) | | |
| PVP K-30 | 2.6 | 9.0 |
| Tween 80 | 5.7 | 19.8 |
| Avicel ™ PH-200 | 6.5 | 22.5 |
| (extragranular) | | |
| Total | 100 | 345.0 |

60. The formulation of claim 1, wherein the formulation comprises:

| | % w/w | mg/cap |
|---|---|---|
| Active Compound of | 26.1 | 120.0 |
| formula (I) | | |
| Avicel ™ PH-200 | 52.8 | 243.0 |
| AcDiSol | 3.6 | 16.6 |
| (intragranular) | | |
| PVP K-30 | 2.6 | 11.9 |
| Tween 80 | 6.9 | 31.7 |
| AcDiSol (extragranular) | 8.0 | 36.8 |
| Total | 100 | 460.0 |

61. The formulation according to claim 1, wherein the formulation is prepared by granulation.

62. A method of making a pharmaceutical formulation comprising granulating a mixture of a compound of formula (I): 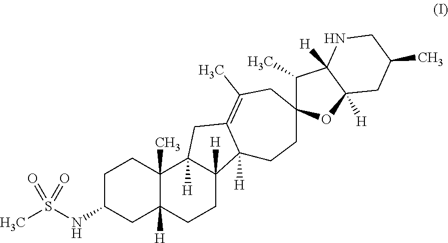
or a pharmaceutically acceptable salt thereof, and a liquid.

63. The method of claim 62, wherein the liquid comprises water.

64. The method of claim 63, wherein the liquid is an aqueous solution of a surfactant.

65. The method according to claim 62, wherein the ratio of the weight of the liquid to the weight of the granulation is greater than 0.25.

66. The method according to claim 62, wherein the method further comprises granulating a filler in the mixture.

67. The method according to claim 62, wherein the method further comprises granulating a binder in the mixture.

68. The method according to claim 62, further comprising the step of drying the granulation.

69. A method of treating cancer comprising orally administering a pharmaceutical formulation as claimed in claim 1 to a patient in need thereof.

70. The method of claim 69, wherein the method further comprises the step of administering one or more other cancer therapeutic agents.
